(19) United States
(12) Patent Application Publication
(10) Pub. No.: US 2022/0106328 A1
(43) Pub. Date: Apr. 7, 2022

(54) HETEROCYCLIC COMPOUNDS

(71) Applicant: Hoffmann-La Roche Inc., Little Falls, NJ (US)

(72) Inventors: Joerg BENZ, Rheinfelden (DE); Luca GOBBI, Buus (CH); Uwe GRETHER, Efringen-Kirchen (DE); Benoit HORNSPERGER, Altkirch (FR); Carsten KROLL, Basel (CH); Bernd KUHN, Reinach (CH); Rainer E. MARTIN, Basel (CH); Fionn O`HARA, Basel (CH); Bernd PUELLMANN, Aesch (CH); Hans RICHTER, Grenzach-Wyhlen (DE); Martin RITTER, Mumpf (CH)

(73) Assignee: Hoffmann-La Roche Inc., Little Falls, NJ (US)

(21) Appl. No.: 17/552,816

(22) Filed: Dec. 16, 2021

Related U.S. Application Data
(63) Continuation of application No. 17/027,952, filed on Sep. 22, 2020.

(30) Foreign Application Priority Data
Sep. 24, 2019 (EP) 19199122.3

(51) Int. Cl. C07D 498/04; A61K 9/20; A61K 9/48

(52) U.S. Cl. CPC C07D 498/04; A61K 9/2009; A61K 9/2054; A61K 9/4866; A61K 9/485; A61K 9/4858; A61K 9/2059

(57) ABSTRACT

The invention provides new heterocyclic compounds having the general formulae (Ia) and (Ib)

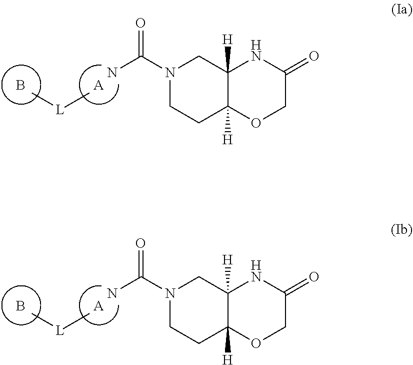

wherein A, B, and L are as described herein, compositions including the compounds, processes of manufacturing the compounds and methods of using the compounds.

### CROSS-REFERENCE TO RELATED APPLICATIONS

This application is a continuation of U.S. application Ser. No. 17/027,952, filed Sep. 22, 2020, which claims priority to EP Application No. 19199122.3, filed Sep. 24, 2019, the disclosures of which are incorporated herein by reference in their entireties.

### FIELD OF THE INVENTION

The present invention relates to organic compounds useful for therapy or prophylaxis in a mammal, and in particular to monoacylglycerol lipase (MAGL) inhibitors for the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine, depression, inflammatory bowel disease, abdominal pain, abdominal pain associated with irritable bowel syndrome and/or visceral pain in a mammal.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28.). In the brain, the main endocannabinoid, 2-arachidonoylglycerol (2-AG), is produced by diacyglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809.). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798.). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclo-oxygenase which produces PGs and the 5-lipoxygenase which produces LTs. Of the various cyclooxygenase products formed during inflammation, PGE2 is one of the most important. These products have been detected at sites of inflammation, e.g. in the cerebrospinal fluid of patients suffering from neurodegenerative disorders and are believed to contribute to inflammatory response and disease progression.

Mice lacking MAGL (Mgll−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2), F2 (PGF2), and thromboxane B2 (TXB2), are strongly decreased. Phospholipase A₂ (PLA₂) enzymes have been viewed as the principal source of AA, but cPLA₂-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis, Alzheimer's disease, Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process. The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a), IL-1b, IL-6, and tumor necrosis factor-a (TNF-a) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and multiple sclerosis (Lleo, A., Cell Mol Life Sci. 2007, 64, 1403). Importantly, genetic and/or pharmacological disruption of MAGL activity also blocks LPS-induced activation of microglial cells in the brain (Nomura, D. K., et al., Science 2011, 334, 809).

In addition, genetic and/or pharmacological disruption of MAGL activity was shown to be protective in several animal models of neurodegeneration including, but not restricted to, Alzheimer's disease, Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska, B., et al., J. Pharmacol. Exp. Ther. 2015, 353, 424) and on mental disorders, such as depression in chronic stress models (Zhong, P., et al., Neuropsychopharmacology 2014, 39, 1763).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bernal-Chico, A., et al., Glia 2015, 63, 163). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421). Inhibition of MAGL was also shown to promote remyelination and functional recovery in a mouse model of progressive multiple sclerosis (Feliu, A., et al., Journal of Neuroscience 2017, 37, 8385).

In addition, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumourigenesis, including in glioblastoma (Qin, H., et al., Cell Biochem. Biophys. 2014, 70, 33; Nomura D K et al., Cell 2009, 140(1), 49-61; Nomura D K et al., Chem. Biol. 2011, 18(7), 846-856, Jinlong Yin et al, Nature Communications 2020, 11, 2978).

The endocannabinoid system is also involved in many gastrointestinal physiological and physiopathological actions (Marquez L. et al., PLoS One 2009, 4(9), e6893). All these effects are driven mainly via cannabinoid receptors (CBRs), CB1 and CB2. CB1 receptors are present throughout the GI tract of animals and healthy humans, especially in the enteric nervous system (ENS) and the epithelial lining, as well as smooth muscle cells of blood vessels in the colonic wall (Wright K. et al., Gastroenterology 2005, 129(2), 437-453; Duncan, M. et al., Aliment Pharmacol Ther 2005, 22(8), 667-683). Activation of CB1 produces anti-emetic, anti-motility, and anti-inflammatory effect, and help to modulate pain (Perisetti, A. et al., Ann Gastroenterol 2020, 33(2), 134-144). CB2 receptors are expressed in immune cells such as plasma cells and macrophages, in the lamina propria of the GI tract (Wright K. et al., Gastroenterology 2005, 129(2), 437-453), and primarily on the epithelium of human colonic tissue associated with inflammatory bowel disease (IBD). Activation of CB2 exerts anti-inflammatory effect by reducing pro-inflammatory cytokines. Expression of MAGL is increased in colonic tissue in UC patients (Marquez L. et al., PLoS One 2009, 4(9), e6893) and 2-AG levels are increased in plasma of IBD patients (Grill, M. et al., Sci Rep 2019, 9(1), 2358). Several animal studies have demonstrated the potential of MAGL inhibitors for symptomatic treatment of IBD. MAGL inhibition prevents TNBS-induced mouse colitis and decreases local and circulating inflammatory markers via a CB1/CB2 MoA (Marquez L. et al., PLoS One 2009, 4(9), e6893). Furthermore, MAGL inhibition improves gut wall integrity and intestinal permeability via a CB1 driven MoA (Wang, J. et al., Biochem Biophys Res Commun 2020, 525(4), 962-967).

In conclusion, suppressing the action and/or the activation of MAGL is a promising new therapeutic strategy for the treatment or prevention of neuroinflammation, neurodegenerative diseases, pain, cancer, mental disorders, inflammatory bowel disease, abdominal pain and abdominal pain associated with irritable bowel syndrome. Furthermore, suppressing the action and/or the activation of MAGL is a promising new therapeutic strategy for providing neuroprotection and myelin regeneration. Accordingly, there is a high unmet medical need for new MAGL inhibitors.

### SUMMARY OF THE INVENTION

In a first aspect, the present invention provides new heterocyclic compounds having the general formulae (Ia) and (Ib)

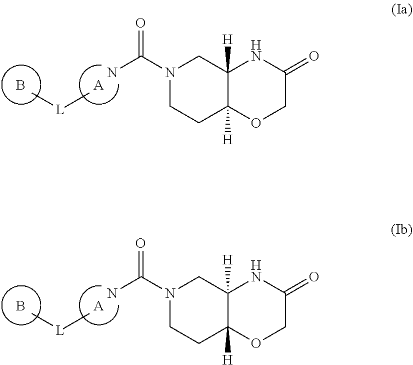

or pharmaceutically acceptable salts thereof, wherein A, B, and L are as described herein.

In a further aspect, the present invention provides a process of manufacturing the urea compounds of formula (Ia) or (Ib) described herein, comprising:
reacting a first amine 4a,5,6,7,8,8a-hexahydro-4H-pyrido[4,3-b][1,4]oxazin-3-one (1)

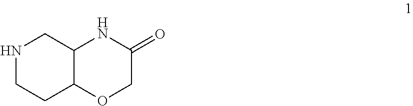 [SMILES: O=C1COC2CCNCC2N1]

with a second amine of formula 2, wherein A, L, and B are as described herein

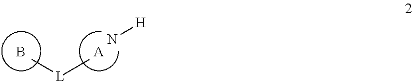

in the presence of a base and a urea forming reagent,
   to form said compound of formula (Ia) or (Ib).

In a further aspect, the present invention provides a compound of formula (Ia) or (Ib) as described herein, when manufactured according to the processes described herein.

In a further aspect, the present invention provides a compound of formula (Ia) or (Ib) as described herein, for use as therapeutically active substance.

In a further aspect, the present invention provides a pharmaceutical composition comprising a compound of formula (Ia) or (Ib) as described herein and a therapeutically inert carrier.

In a further aspect, the present invention provides the use of a compound of formula (Ia) or (Ib) as described herein or of a pharmaceutical composition described herein for inhibiting monoacylglycerol lipase (MAGL) in a mammal.

In a further aspect, the present invention provides the use of a compound of formula (I) as described herein or of a pharmaceutical composition described herein for the treatment or prophylaxis of neuroinflammation, neurodegenerative diseases, pain, cancer, mental disorders and/or inflammatory bowel disease in a mammal.

In a further aspect, the present invention provides the use of a compound of formula (I) as described herein or of a pharmaceutical composition described herein for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl are methyl and tert-butyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C₁₋₆-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cyano” refers to a —CN (nitrile) group.

The term “hydroxy” refers to an —OH group.

The term “alkylsulfonyl” refers to an alkyl group, as previously defined, attached to the parent molecular moiety via an SO₂ group.

The term “carbamoyl” refers to a group H₂N—C(O)—.

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms, most preferably 1 hydrogen atom of the alkyl group have been replaced by a hydroxy group. Preferred, yet non-limiting examples of hydroxyalkyl are hydroxymethyl and hydroxyethyl (e.g. 2-hydroxyethyl). A particularly preferred, yet non-limiting example of hydroxyalkyl is 2-hydroxyethyl.

The term “alkoxyalkyl” refers to an alkyl group, wherein at least one of the hydrogen atoms of the alkyl group has been replaced by a alkoxy group. Preferably, “alkoxyalkyl” refers to an alkyl group wherein 1, 2 or 3 hydrogen atoms, most preferably 1 hydrogen atom of the alkyl group have been replaced by an alkoxy group. A preferred, yet non-limiting example of alkoxyalkyl is 2-methoxyethyl.

The term “cycloalkyl” as used herein refers to a saturated or partly unsaturated monocyclic or bicyclic hydrocarbon group of 3 to 10 ring carbon atoms (“C₃-C₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl and cycloheptyl.

The terms “heterocyclyl” and “heterocycloalkyl” are used herein interchangeably and refer to a saturated or partly unsaturated mono- or bicyclic, preferably monocyclic ring system of 3 to 14 ring atoms, preferably 4 to 7 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. More preferably, one of said ring atoms is N, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidinyl, pyrrolidinyl, piperidyl, morpholinyl, 2-azaspiro[3.3]heptanyl, and 2,3,3a,4,6,6a-hexahydro-1H-pyrrolo[3,4-c]pyrrolyl. Preferred, yet non-limiting examples of heterocyclyl include azetidin-1-yl, 2-azaspiro[3.3]heptan-2-yl, 2,3,3a,4,6,6a-hexahydro-1H-pyrrolo[3,4-c]pyrrol-5-yl, and 7-azaspiro[3.5]nonan-7-yl. A particularly preferred, yet non-limiting example of heterocyclyl includes azetidin-1-yl.

The term “aryl” refers to a monocyclic, bicyclic, or tricyclic carbocyclic ring system having a total of 6 to 14 ring members, preferably, 6 to 12 ring members, and more preferably 6 to 10 ring members, and wherein at least one ring in the system is aromatic. Some non-limiting examples of aryl include phenyl and 9H-fluorenyl (e.g., 9H-fluoren-9-yl). A particularly preferred, yet non-limiting example of aryl is phenyl.

The term “heteroaryl” refers to a mono- or multivalent, monocyclic or bicyclic, preferably monocyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include 2-pyridyl, 3-pyridyl, 4-pyridyl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl and oxazol-5-yl. Particularly preferred, yet non-limiting examples of heteroaryl are pyridyl, in particular 3-pyridyl, and oxazolyl, in particular oxazol-2-yl.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl (CF₃) and trifluoroethyl (e.g., 2,2,2-trifluoroethyl).

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. A particularly preferred, yet non-limiting example of haloalkoxy is trifluoromethoxy (—OCF₃).

The term “haloaryl” refers to an aryl group, wherein at least one of the hydrogen atoms of the aryl group has been replaced by a halogen atom, preferably fluoro or chloro. Preferably, “haloaryl” refers to an aryl group wherein 1, 2 or 3 hydrogen atoms of the aryl group have been replaced by a halogen atom, most preferably fluoro. A particularly preferred, yet non-limiting example of haloaryl is 4-fluorophenyl.

The term “aryloxy” refers to an aryl group, as previously defined, attached to the parent molecular moiety via an oxygen atom. A preferred, yet non-limiting example of aryloxy is phenoxy.

The term “cycloalkyloxy” refers to a cycloalkyl group, as previously defined, attached to the parent molecular moiety via an oxygen atom. A preferred, yet non-limiting example of cycloalkyloxy is cyclopropoxy.

The term “heteroaryloxy” refers to a heteroaryl group, as previously defined, attached to the parent molecular moiety via an oxygen atom. A preferred, yet non-limiting example of heteroaryloxy is pyridyloxy (e.g., 2-pyridiyloxy).

The term “heterocyclyloxy” refers to a heterocyclyl group, as previously defined, attached to the parent molecular moiety via an oxygen atom. A preferred, yet non-limiting example of heterocyclyloxy is pyrrolidinyloxy (e.g., pyrrolidi-3-yl-oxy).

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (Ia) or (Ib) are hydrochloride salts.

The term “protective group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, New York.

The term “urea forming reagent” refers to a chemical compound that is able to render a first amine to a species that will react with a second amine, thereby forming an urea derivative. Non-limiting examples of urea forming reagents include bis(trichloromethyl) carbonate, phosgene, trichloromethyl chloroformate, (4-nitrophenyl)carbonate and 1,1′-carbonyldiimidazole. The urea forming reagents described in Sartori, G., et al., Green Chemistry 2000, 2, 140 are incorporated herein by reference.

The compounds of formula (Ia) or (Ib) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g., arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain including chemotherapy induced neuropathy, phantom pain and phsychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy. In one embodiment, “pain” is chemotherapy induced neuropathy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “cancer” refers to a disease characterized by the presence of a neoplasm or tumor resulting from abnormal uncontrolled growth of cells (such cells being “cancer cells”). As used herein, the term cancer explicitly includes, but is not limited to, hepatocellular carcinoma, colon carcinogenesis and ovarian cancer.

The term “mammal” as used herein includes both humans and non-humans and includes but is not limited to humans, non-human primates, canines, felines, murines, bovines, equines, and porcines. In a particularly preferred embodiment, the term “mammal” refers to humans.

### Compounds of the Invention

In a first aspect (A1), the present invention provides compounds of formula (Ia) or (Ib)

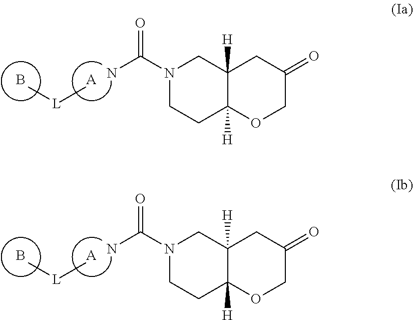

or a pharmaceutically acceptable salt thereof, wherein:
A is a 3-14 membered heterocycle substituted with R^{A};
B is C₆-C₁₄-aryl or 5-14 membered heteroaryl substituted with R¹, R² and R³;
L is selected from a covalent bond, —C≡C—, —CHR^{L}—, —CH₂CHR^{L}—, —O—, —OCH₂—, and —CH₂O—; and
R¹, R², and R³ are independently selected from hydrogen, halogen, cyano, C₁-C₆-alkylsulfonyl, R^{b}R^{c}N, C₁-C₆-alkyl, C₁-C₆-alkoxy, halo-C₁-C₆-alkyl, halo-C₁-C₆-alkoxy, hydroxy-C₁-C₆-alkyl, C₆-C₁₄-aryl, C₃-C₁₀-cycloalkyl, 3-14 membered heterocyclyl, 5-14 membered heteroaryl, C₆-C₁₄-aryloxy, C₃-C₁₀-cycloalkyloxy, 3-14 membered heterocyclyloxy, and 5-14 membered heteroaryloxy, wherein said C₃-C₁₀-cycloalkyl, C₆-C₁₄-aryl, 3-14 membered heterocyclyl, 5-14 membered heteroaryl, C₆-C₁₄-aryloxy, C₃-C₁₀-cycloalkyloxy, 3-14 membered heterocyclyloxy, and 5-14 membered heteroaryloxy are optionally substituted with one or more substituents that are independently selected from halogen, C₁-C₆-alkyl, halo-C₁-C₆-alkyl, C₁-C₆-alkoxy, halo-C₁-C₆-alkoxy, and carbamoyl;
R^{A} is selected from hydrogen and C₁-C₆-alkyl;
R^{b} and R^{c} are independently selected from hydrogen, C₁-C₆-alkyl and C₆-C₁₄-aryl; and
R^{L} is selected from hydrogen, C₁-C₆-alkyl, hydroxy-C₁-C₆-alkyl, alkoxy-C₁-C₆-alkyl, halo-C₁-C₆-alkyl, C₆-C₁₄-aryl, and halo-C₆-C₁₄-aryl.

The invention also provides the following enumerated Embodiments (E) of the first aspect (A1) of the invention:
E1. The compound of formula (Ia) or (Ib) according to A1, or a pharmaceutically acceptable salt thereof, wherein:
   A is a 3-14 membered heterocycle substituted with R^{A};
   B is C₆-C₁₄-aryl or 5-14 membered heteroaryl substituted with R¹, R² and R³;
   L is selected from a covalent bond, —C≡C—, —CHR^{L}—, —CH₂CHR^{L}—, —O—, —OCH₂—, and —CH₂O—; and
   R¹, R², and R³ are independently selected from hydrogen, halogen, cyano, C₁-C₆-alkylsulfonyl, R^{b}R^{c}N, C₁-C₆-alkyl, C₁-C₆-alkoxy, halo-C₁-C₆-alkyl, halo-C₁-C₆-alkoxy, C₆-C₁₄-aryl, C₃-C₁₀-cycloalkyl, 3-14 membered heterocyclyl, 5-14 membered heteroaryl, C₆-C₁₄-aryloxy, C₃-C₁₀-cycloalkyloxy, 3-14 membered heterocyclyloxy, and 5-14 membered heteroaryloxy, wherein said C₃-C₁₀-cycloalkyl, C₆-C₁₄-aryl, 3-14 membered heterocyclyl, 5-14 membered heteroaryl, C₆-C₁₄-aryloxy, C₃-C₁₀-cycloalkyloxy, 3-14 membered heterocyclyloxy, and 5-14 membered heteroaryloxy are optionally substituted with one or more substituents that are independently selected from halogen, C₁-C₆-alkyl, halo-C₁-C₆-alkyl, C₁-C₆-alkoxy, halo-C₁-C₆-alkoxy, and carbamoyl;
   R^{A} is selected from hydrogen and C₁-C₆-alkyl;
   R^{b} and R^{c} are independently selected from hydrogen, C₁-C₆-alkyl and C₆-C₁₄-aryl; and
   R^{L} is selected from hydrogen, C₁-C₆-alkyl, hydroxy-C₁-C₆-alkyl, alkoxy-C₁-C₆-alkyl, halo-C₁-C₆-alkyl, C₆-C₁₄-aryl, and halo-C₆-C₁₄-aryl.
E2. The compound of formula (Ia) or (Ib) according to A1 or E1, or a pharmaceutically acceptable salt thereof, wherein
   A is a 3-14 membered heterocycle;
   B is C₆-C₁₄-aryl substituted with R¹, R² and R³;
   L is selected from a covalent bond, —CH₂—, —CH₂CH₂—, —OCH₂—, and —CH₂O—; and
   R¹, R², and R³ are independently selected from hydrogen, halogen, C₁-C₆-alkyl, C₁-C₆-alkoxy, halo-C₁-C₆-alkyl, halo-C₁-C₆-alkoxy, C₆-C₁₄-aryloxy, C₆-C₁₄-aryl, and C₃-C₁₀-cycloalkyl, wherein said C₃-C₁₀-cycloalkyl and C₆-C₁₄-aryl are optionally substituted with one or more substituents that are independently selected from halogen and halo-C₁-C₆-alkyl.
E3. The compound of formula (Ia) or (Ib) according to any one of A1 and E1 to E2, or a pharmaceutically acceptable salt thereof, wherein the compound of formula (Ia) or (Ib) is not selected from:
   (4aS,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
   (4aR,8aR)-6-[4-[[4-(trifluoromethyl)phenyl]methyl]piperidine-1-carbonyl]-4,4a,5,7,8,8a-hexahydropyrido[4,3-b][1,4]oxazin-3-one; and
   rac-(4aS,8aS)-6-[4-(2-methylallyl)piperidine-1-carbonyl]-4,4a,5,7,8,8a-hexahydropyrido[4,3-b][1,4]oxazin-3-one (CAS 1941372-36-6).
E4. The compound of formula (Ia) or (Ib) according to any one of A1 and E1 to E3, or a pharmaceutically acceptable salt thereof, wherein A is a 4-9 membered heterocycle, wherein 1, 2, or 3 of the ring atoms are heteroatoms selected from N, O and S, the remaining ring atoms being carbon.
E5. The compound of formula (Ia) or (Ib) according to any one of A1 and E1 to E3, or a pharmaceutically acceptable salt thereof, wherein A is a 4-9 membered heterocycle, wherein one of the ring atoms is nitrogen, the remaining ring atoms being carbon.
E6. The compound of formula (Ia) or (Ib) according to any one of A1 and E1 to E3, or a pharmaceutically acceptable salt thereof, wherein A is a 4-9 membered heterocycle selected from azetidin-1-yl, 2-azaspiro[3.3]heptan-2-yl, 2,3,3a,4,6,6a-hexahydro-1H-pyrrolo[3,4-c]pyrrol-5-yl, and 7-azaspiro[3.5]nonan-7-yl.
E7. The compound of formula (Ia) or (Ib) according to any one of A1 and E1 to E3, or a pharmaceutically acceptable salt thereof, wherein A is a 4-8 membered heterocycle, wherein 1, 2, or 3 of the ring atoms are heteroatoms selected from N, O and S, the remaining ring atoms being carbon.
E8. The compound of formula (Ia) or (Ib) according to any one of A1 and E1 to E3, or a pharmaceutically acceptable salt thereof, wherein A is a 4-8 membered heterocycle, wherein one of the ring atoms is nitrogen, the remaining ring atoms being carbon.
E9. The compound of formula (Ia) or (Ib) according to any one of A1 and E1 to E3, or a pharmaceutically acceptable salt thereof, wherein A is a 4-8 membered heterocycle selected from azetidin-1-yl, 2-azaspiro[3.3]heptan-2-yl, and 2,3,3a,4,6,6a-hexahydro-1H-pyrrolo[3,4-c]pyrrol-5-yl.
E10. The compound of formula (Ia) or (Ib) according to any one of A1 and E1 to E3, or a pharmaceutically acceptable salt thereof, wherein A is azetidine or 7-azaspiro[3.5]nonan-7-yl.
E11. The compound of formula (Ia) or (Ib) according to any one of A1 and E1 to E3, or a pharmaceutically acceptable salt thereof, wherein A is azetidine.
E12. The compound of formula (Ia) or (Ib) according to any one of A1 and E1 to E11, or a pharmaceutically acceptable salt thereof, wherein R^{A} is hydrogen.
E13. The compound of formula (Ia) or (Ib) according to any one of A1 and E1 to E12, or a pharmaceutically acceptable salt thereof, wherein B is phenyl substituted with R¹, R² and R³.
E14. The compound of formula (Ia) or (Ib) according to any one of A1 and E1 to E13, or a pharmaceutically acceptable salt thereof, wherein:
   L is selected from a covalent bond, —CHR^{L}—, —CH₂CH₂—, —O—, —OCH₂—, and —CH₂O—; and
   R^{L} is hydrogen or halo-C₆-C₁₄-aryl.
E15. The compound of formula (Ia) or (Ib) according to any one of A1 and E1 to E13, or a pharmaceutically acceptable salt thereof, wherein L is selected from a covalent bond, —O—, —CH₂—, —CH₂CH₂—, —OCH₂—, and —CH₂O—.
E16. The compound of formula (Ia) or (Ib) according to any one of A1 and E1 to E13, or a pharmaceutically acceptable salt thereof, wherein L is selected from a covalent bond, —CH₂—, —CH₂CH₂—, —OCH₂—, and —CH₂O—.
E17. The compound of formula (Ia) or (Ib) according to any one of A1 and E1 to E13, or a pharmaceutically acceptable salt thereof, wherein L is selected from a covalent bond, —CH₂CH₂—, and —CH₂O—.
E18. The compound of formula (Ia) or (Ib) according to any one of A1 and E1 to E13, or a pharmaceutically acceptable salt thereof, wherein L is a covalent bond or —O—.
E19. The compound of formula (Ia) or (Ib) according to any one of A1 and E1 to E13, or a pharmaceutically acceptable salt thereof, wherein L is a covalent bond.
E20. The compound of formula (Ia) or (Ib) according to any one of A1 and E1 to E19, or a pharmaceutically acceptable salt thereof, wherein R¹ is selected from C₁-C₆-alkyl, halo-C₁-C₆-alkyl, halo-C₁-C₆-alkoxy, C₆-C₁₄-aryloxy, C₆-C₁₄-aryl, and C₃-C₁₀-cycloalkyl, wherein said C₃-C₁₀-cycloalkyl and C₆-C₁₄-aryl are substituted with 1-2 substituents that are independently selected from halogen and halo-C₁-C₆-alkyl.
E21. The compound of formula (Ia) or (Ib) according to any one of A1 and E1 to E19, or a pharmaceutically acceptable salt thereof, wherein R¹ is selected from halogen, C₁-C₆-alkyl, halo-C₁-C₆-alkyl, halo-C₁-C₆-alkoxy, hydroxy-C₁-C₆-alkyl, C₆-C₁₄-aryloxy, C₆-C₁₄-aryl, and C₃-C₁₀-cycloalkyl, wherein said C₃-C₁₀-cycloalkyl, C₆-C₁₄-aryloxy and C₆-C₁₄-aryl are substituted with 1-2 substituents that are independently selected from halogen and halo-C₁-C₆-alkyl.
E22. The compound of formula (Ia) or (Ib) according to any one of A1 and E1 to E19, or a pharmaceutically acceptable salt thereof, wherein R¹ is selected from C₆-C₁₄-aryloxy and C₃-C₁₀-cycloalkyl substituted with halo-C₁-C₆-alkyl.
E23. The compound of formula (Ia) or (Ib) according to any one of A1 and E1 to E19, or a pharmaceutically acceptable salt thereof, wherein R¹ is selected from C₆-C₁₄-aryloxy, halo-C₁-C₆-alkyl and C₃-C₁₀-cycloalkyl substituted with halo-C₁-C₆-alkyl.
E24. The compound of formula (Ia) or (Ib) according to any one of A1 and E1 to E19, or a pharmaceutically acceptable salt thereof, wherein R¹ is selected from phenoxy and (trifluoromethyl)cyclopropyl.
E25. The compound of formula (Ia) or (Ib) according to any one of A1 and E1 to E19, or a pharmaceutically acceptable salt thereof, wherein R¹ is selected from phenoxy, CF₃ and (trifluoromethyl)cyclopropyl.
E26. The compound of formula (Ia) or (Ib) according to any one of A1 and E1 to E25, or a pharmaceutically acceptable salt thereof, wherein R² is selected from hydrogen and halogen.
E27. The compound of formula (Ia) or (Ib) according to any one of A1 and E1 to E25, or a pharmaceutically acceptable salt thereof, wherein R² is selected from hydrogen and fluoro.
E28. The compound of formula (Ia) or (Ib) according to any one of A1 and E1 to E25, or a pharmaceutically acceptable salt thereof, wherein R² is hydrogen.
E29. The compound of formula (Ia) or (Ib) according to any one of A1 and E1 to E28, or a pharmaceutically acceptable salt thereof, wherein R³ is hydrogen.
E30. The compound of formula (Ia) or (Ib) according to any one of A1 and E1 to E29, or a pharmaceutically acceptable salt thereof, wherein:
   B is C₆-C₁₄-aryl substituted with R¹ and R²;
   R¹ is selected from C₁-C₆-alkyl, halo-C₁-C₆-alkyl, halo-C₁-C₆-alkoxy, C₆-C₁₄-aryloxy, C₆-C₁₄-aryl, and C₃-C₁₀-cycloalkyl, wherein said C₃-C₁₀-cycloalkyl and C₆-C₁₄-aryl are substituted with 1-2 substituents that are independently selected from halogen and halo-C₁-C₆-alkyl; and
   R² is selected from hydrogen and halogen.
E31. The compound of formula (Ia) or (Ib) according to any one of A1 and E1 to E29, or a pharmaceutically acceptable salt thereof, wherein:
   B is C₆-C₁₄-aryl substituted with R¹ and R²;
   R¹ is selected from halogen, C₁-C₆-alkyl, halo-C₁-C₆-alkyl, halo-C₁-C₆-alkoxy, hydroxy-C₁-C₆-alkyl, C₆-C₁₄-aryloxy, C₆-C₁₄-aryl, and C₃-C₁₀-cycloalkyl, wherein said C₃-C₁₀-cycloalkyl, C₆-C₁₄-aryloxy and C₆-C₁₄-aryl are substituted with 1-2 substituents that are independently selected from halogen and halo-C₁-C₆-alkyl; and
   R² is selected from hydrogen and halogen.
E32. The compound of formula (Ia) or (Ib) according to any one of A1 and E1 to E29, or a pharmaceutically acceptable salt thereof, wherein:
   B is C₆-C₁₄-aryl substituted with R¹; and
   R¹ is selected from C₆-C₁₄-aryloxy and C₃-C₁₀-cycloalkyl substituted with halo-C₁-C₆-alkyl.
E33. The compound of formula (Ia) or (Ib) according to any one of A1 and E1 to E29, or a pharmaceutically acceptable salt thereof, wherein:
   B is C₆-C₁₄-aryl substituted with R¹ and R²;
   R¹ is selected from C₆-C₁₄-aryloxy, halo-C₁-C₆-alkyl and C₃-C₁₀-cycloalkyl substituted with halo-C₁-C₆-alkyl; and
   R² is selected from hydrogen and halogen.
E34. The compound of formula (Ia) or (Ib) according to any one of A1 and E1 to E29, or a pharmaceutically acceptable salt thereof, wherein:
   B is phenyl substituted with R¹; and
   R¹ is selected from phenoxy and (trifluoromethyl)cyclopropyl.
E35. The compound of formula (Ia) or (Ib) according to any one of A1 and E1 to E29, or a pharmaceutically acceptable salt thereof, wherein:
   B is phenyl substituted with R¹ and R²;
   R¹ is selected from phenoxy, CF₃ and (trifluoromethyl)cyclopropyl; and
   R² is hydrogen or fluoro.
E36. The compound of formula (Ia) or (Ib) according to any one of A1 and E1 to E3, or a pharmaceutically acceptable salt thereof, wherein:
   A is a 3-14 membered heterocycle;
   B is C₆-C₁₄-aryl substituted with R¹ and R²;
   L is selected from a covalent bond, —CH₂CH₂—, —CHR^{L}—, —O— and —CH₂O—;
   R^{L} is hydrogen or halo-C₆-C₁₄-aryl;
   R¹ is selected from halogen, C₁-C₆-alkyl, halo-C₁-C₆-alkyl, halo-C₁-C₆-alkoxy, hydroxy-C₁-C₆-alkyl, C₆-C₁₄-aryloxy, C₆-C₁₄-aryl, and C₃-C₁₀-cycloalkyl, wherein said C₃-C₁₀-cycloalkyl, C₆-C₁₄-aryloxy and C₆-C₁₄-aryl are substituted with 1-2 substituents that are independently selected from halogen and halo-C₁-C₆-alkyl; and
   R² is selected from hydrogen and halogen.
E37. The compound of formula (Ia) or (Ib) according to any one of A1 and E1 to E3, or a pharmaceutically acceptable salt thereof, wherein:
   A is a 3-14 membered heterocycle;
   B is C₆-C₁₄-aryl substituted with R¹ and R²;
   L is selected from a covalent bond, —CH₂CH₂—, and —CH₂O—;
   R¹ is selected from C₁-C₆-alkyl, halo-C₁-C₆-alkyl, halo-C₁-C₆-alkoxy, C₆-C₁₄-aryloxy, C₆-C₁₄-aryl, and C₃-C₁₀-cycloalkyl, wherein said C₃-C₁₀-cycloalkyl and C₆-C₁₄-aryl are substituted with 1-2 substituents that are independently selected from halogen and halo-C₁-C₆-alkyl; and
   R² is selected from hydrogen and halogen.
E38. The compound of formula (Ia) or (Ib) according to any one of A1 and E1 to E3, or a pharmaceutically acceptable salt thereof, wherein:
   A is a 3-14 membered heterocycle;
   B is C₆-C₁₄-aryl substituted with R¹ and R²;
   L is a covalent bond or —O—;
   R¹ is selected from C₆-C₁₄-aryloxy, halo-C₁-C₆-alkyl and C₃-C₁₀-cycloalkyl substituted with halo-C₁-C₆-alkyl; and
   R² is hydrogen or halogen.
E39. The compound of formula (Ia) or (Ib) according to any one of A1 and E1 to E3, or a pharmaceutically acceptable salt thereof, wherein:
   A is a 3-14 membered heterocycle;
   B is C₆-C₁₄-aryl substituted with R¹;
   L is a covalent bond; and
   R¹ is selected from C₆-C₁₄-aryloxy and C₃-C₁₀-cycloalkyl substituted with halo-C₁-C₆-alkyl.
E40. The compound of formula (Ia) or (Ib) according to any one of A1 and E1 to E3, or a pharmaceutically acceptable salt thereof, wherein:
   A is azetidine or 7-azaspiro[3.5]nonan-7-yl;
   B is phenyl substituted with R¹ and R²;
   L is selected from a covalent bond, —CH₂— or —O—;
   R¹ is selected from phenoxy, CF₃ and (trifluoromethyl)cyclopropyl; and
   R² is hydrogen or fluoro.
E41. The compound of formula (Ia) or (Ib) according to any one of A1 and E1 to E3, or a pharmaceutically acceptable salt thereof, wherein:
   A is azetidine;
   B is phenyl substituted with R¹;
   L is a covalent bond; and
   R¹ is selected from phenoxy and (trifluoromethyl)cyclopropyl.
E42. The compound of formula (Ia) or (Ib) according to any one of A1 and E1 to E41, or a pharmaceutically acceptable salt thereof, selected from the compounds disclosed in Table 1.
E43. The compound of formula (Ia) or (Ib) according to any one of A1 and E1 to E41, or a pharmaceutically acceptable salt thereof, selected from:
   (4aR,8aR)-6-[3-[4-[1-(trifluoromethyl)cyclopropyl]phenyl]azetidine-1-carbonyl]-4,4a,5,7,8,8a-hexahydropyrido[4,3-b][1,4]oxazin-3-one

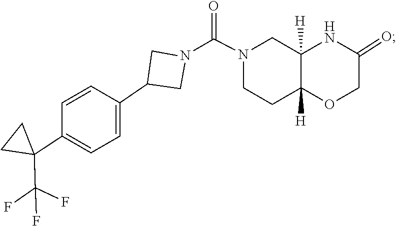 [SMILES: O=C1CO[C@@H]2CCN(C(=O)N3CC(c4ccc(C5(C(F)(F)F)CC5)cc4)C3)C[C@H]2N1]

(4aS,8aS)-6-(3-(4-phenoxyphenyl)azetidine-1-carbonyl)hexahydro-2H-pyrido[4,3-b][1,4]oxazin-3(4H)-one

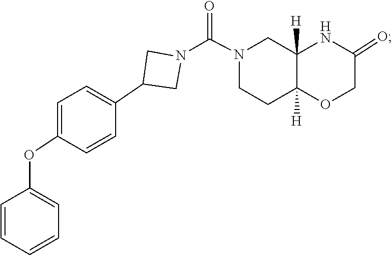 [SMILES: O=C1CO[C@H]2CCN(C(=O)N3CC(c4ccc(Oc5ccccc5)cc4)C3)C[C@@H]2N1]

and
(−)- or (+)-trans-6-[2-[2-Fluoro-4-(trifluoromethyl)phenoxy]-7-azaspiro[3.5]nonane-7-carbonyl]-4,4a,5,7,8,8a-hexahydropyrido[4,3-b][1,4]oxazin-3-one

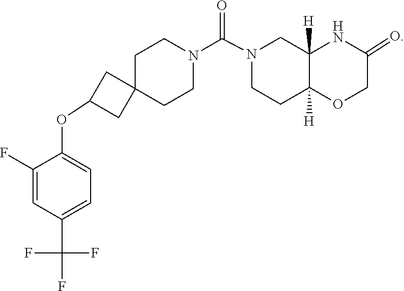 [SMILES: O=C1CO[C@H]2CCN(C(=O)N3CCC4(CC3)CC(Oc3ccc(C(F)(F)F)cc3F)C4)C[C@@H]2N1]

E44. The compound of formula (Ia) or (Ib) according to any one of A1 and E1 to E41, or a pharmaceutically acceptable salt thereof, selected from:
   (4aR,8aR)-6-[3-[4-[1-(trifluoromethyl)cyclopropyl]phenyl]azetidine-1-carbonyl]-4,4a,5,7,8,8a-hexahydropyrido[4,3-b][1,4]oxazin-3-one

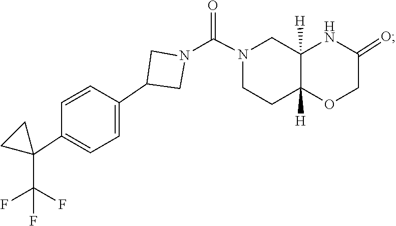 [SMILES: O=C1CO[C@@H]2CCN(C(=O)N3CC(c4ccc(C5(C(F)(F)F)CC5)cc4)C3)C[C@H]2N1]

and
(4aS,8aS)-6-(3-(4-phenoxyphenyl)azetidine-1-carbonyl)hexahydro-2H-pyrido[4,3-b][1,4]oxazin-3(4H)-one

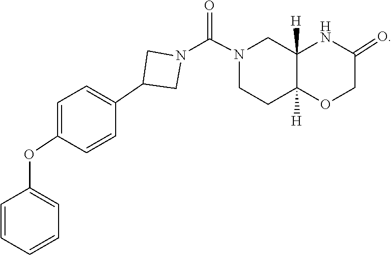 [SMILES: O=C1CO[C@H]2CCN(C(=O)N3CC(c4ccc(Oc5ccccc5)cc4)C3)C[C@@H]2N1]

In a particular embodiment, the present invention provides pharmaceutically acceptable salts of the compounds according to formula (Ia) or (Ib) as described herein. In a further particular embodiment, the present invention provides compounds according to formula (Ia) or (Ib) as described herein as free bases.

In some embodiments, the compounds of formula (Ia) or (Ib) are isotopically-labeled by having one or more atoms therein replaced by an atom having a different atomic mass or mass number. Such isotopically-labeled (i.e., radiolabeled) compounds of formula (Ia) or (Ib) are considered to be within the scope of this disclosure. Examples of isotopes that can be incorporated into the compounds of formula (Ia) or (Ib) include isotopes of hydrogen, carbon, nitrogen, oxygen, phosphorous, sulfur, fluorine, chlorine, and iodine, such as, but not limited to ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (Ia) or (Ib), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (Ia) or (Ib) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (Ia) or (Ib) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

The preparation of compounds of formula (Ia) or (Ib) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (Ia) or (Ib) contain one or more functional groups which are not stable or are reactive under the reaction conditions of one or more reaction steps, appropriate protective groups (as described e.g., in “Protective Groups in Organic Chemistry” by T. W. Greene and P. G. M. Wutts, 5th Ed., 2014, John Wiley & Sons, New York) can be introduced before the critical step applying methods well known in the art. Such protective groups can be removed at a later stage of the synthesis using standard methods described in the literature.

If starting materials or intermediates contain stereogenic centers, compounds of formula (Ia) or (Ib) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent. It is equally possible to separate starting materials and intermediates containing stereogenic centers to afford diastereomerically/enantiomerically enriched starting materials and intermediates. Using such diastereomerically/enantiomerically enriched starting materials and intermediates in the synthesis of compounds of formula (Ia) or (Ib) will typically lead to the respective diastereomerically/enantiomerically enriched compounds of formula (Ia) or (Ib).

A person skilled in the art will acknowledge that in the synthesis of compounds of formula (Ia) or (Ib)—insofar not desired otherwise—an “orthogonal protection group strategy” will be applied, allowing the cleavage of several protective groups one at a time each without affecting other protective groups in the molecule. The principle of orthogonal protection is well known in the art and has also been described in literature (e.g., Barany, G., Merrifield, R. B., J. Am. Chem. Soc. 1977, 99, 7363; Waldmann, H., et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (Ia) or (Ib) can be manufactured by the methods given below, by the methods given in the examples or by analogous methods. Appropriate reaction conditions for the individual reaction steps are known to a person skilled in the art. Also, for reaction conditions described in literature affecting the described reactions see for example: “Comprehensive Organic Transformations: A Guide to Functional Group Preparations”, Richard C. Larock, 2nd Ed., 1999, John Wiley & Sons, N. Y.). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

If starting materials or intermediates are not commercially available or their synthesis not described in literature, they can be prepared in analogy to existing procedures for close analogues or as outlined in the experimental section.

The following abbreviations are used in the present text:

AcOH=acetic acid, Boc=tert-butyloxycarbonyl, CAS RN=chemical abstracts registration number, Cbz=benzyloxycarbonyl, DME=dimethoxyethane, DMF=N,N-dimethylformamide, DIPEA=N,N-diisopropylethylamine, ESI=electrospray ionization, EtOAc=ethyl acetate, EtOH=ethanol, h=hour(s), H₂O=water, HCl=hydrogen chloride, HPLC=high performance liquid chromatography, IPA=2-propanol, K₂CO₃=potassium carbonate, K₃PO₄=potassium phosphate tribasic, LiHMDS=lithium bis(trimethylsilyl)amide, MgSO₄=magnesium sulfate, min=minute(s), mL=milliliter, MPLC=medium pressure liquid chromatography, MS=mass spectrum, NaH=sodium hydride, NaHCO₃=sodium hydrogen carbonate, NaOH=sodium hydroxide, Na₂CO₃=sodium carbonate, Na₂SO₄=sodium sulfate, nBuLi=n-butyllithium, NEt₃=triethylamine (TEA), NH₄Cl=ammonium chloride, OAc=acetoxy, PG=protective group, Pd/C=palladium on activated carbon, Pd(OH)₂=palladium hydroxide, R=any group, rt=room temperature, SFC=supercritical fluid chromatography, TEA=triethylamine, TFA=trifluroacetic acid, THF=tetrahydrofuran.

Compounds of formula I wherein A, B and L are as described herein can be synthesized in analogy to literature procedures and/or as depicted for example in Scheme 1.

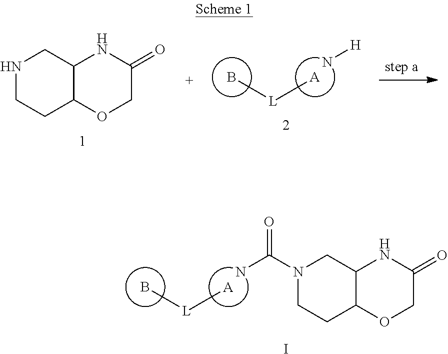

Accordingly, 4a,5,6,7,8,8a-hexahydro-4H-pyrido[4,3-b][1,4]oxazin-3-ones 1 are reacted with intermediates 2 in the presence of a urea forming reagent such as bis(trichloromethyl) carbonate using a suitable base and solvent such as, e.g. sodium bicarbonate in DCM, to give compounds of formula I (step a). Further urea forming reagents include but are not limited to phosgene, trichloromethyl chloroformate, (4-nitrophenyl)carbonate or 1,1′-carbonyldiimidazole. Reactions of this type and the use of these reagents are widely described in literature (e.g. Sartori, G., et al., Green Chemistry 2000, 2, 140). A person skilled in the art will acknowledge that the order of the addition of the reagents can be important in this type of reactions due to the reactivity and stability of the intermediary formed carbamoyl chlorides, as well as for avoiding formation of undesired symmetrical urea by-products.

Intermediates 1 may be synthesized as depicted for example in Scheme 2 and/or in analogy to methods described in literature.

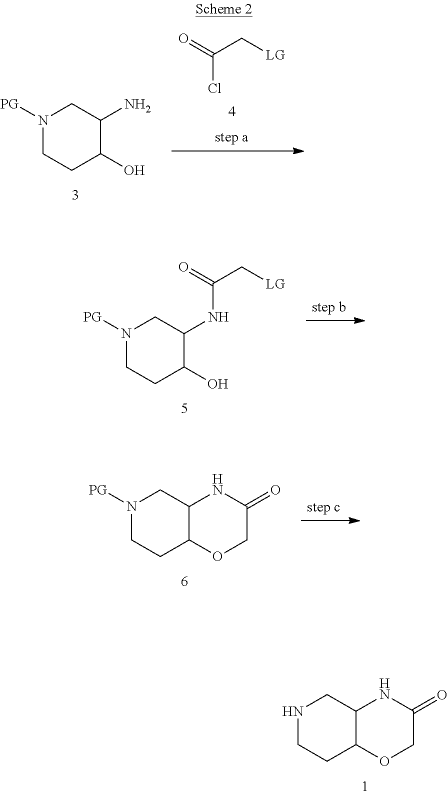

Thus, 3-aminopiperidin-4-ol derivatives 3 in which “PG” signifies a suitable protective group such as a Cbz or Boc protective group can be acylated for example with chloro- or bromoacetyl chloride 4, in which “LG” signifies a suitable leaving group (e.g., Cl or Br), using a suitable base such as sodium or potassium carbonate, sodium hydroxide or sodium acetate in an appropriate solvent such as THF, water, acetone or mixtures thereof, to provide intermediates 5 (step a).

Intermediates 5 can be cyclized to intermediates 6 using methods well known in the art, for example by treatment of 5 with sodium hydride in THF or potassium tert-butoxide in IPA and water (step b). Reactions of that type are described in literature (e.g., Rafinski, Z., et al., J. Org. Chem. 2015, 80, 7468; Dugar, S., et al., Synthesis 2015, 47, 712; WO2005/066187).

Removal of the protective group in intermediates 6, applying methods known in the art (e.g., a Boc group using TFA in DCM, HCl in dioxane or diethylether, or 4-methylbenzenesulfonic acid hydrate in ethyl acetate or mixtures therefore at temperatures between 0° C. and room temperature, a Cbz group using hydrogen in the presence of a suitable catalyst such as Pd or Pd(OH)₂ on charcoal in a suitable solvent such as MeOH, EtOH, ethyl acetate or mixtures therefore and as described for example in “Protective Groups in Organic Chemistry” by T. W. Greene and P. G. M. Wuts, 4th Ed., 2006, Wiley, New York), furnishes intermediates 1 (step c).

Intermediates 1 can be obtained as mixtures of diastereomers and enantiomers, respectively, or as single stereoisomers depending on whether racemic mixtures or enantiomerically pure forms of cis- or trans-3-aminopiperidin-4-ol derivatives 3 are employed in their syntheses. Intermediates 3 are commercially available and their synthesis has also been described in literature (e.g., WO2005/066187; WO2011/0059118; WO2016/185279).

Optically pure trans-configured intermediates 1B and 1C can be obtained for example according to Scheme 3. Chiral separation of appropriately protected rac-trans-4a,5,6,7,8,8a-hexahydro-4H-pyrido[4,3-b][1,4]oxazin-3-one (7) (“PG” signifies a suitable protective group such as a Cbz or Boc) using methods known in the art, e.g. by diastereomeric salt crystallization or by chiral chromatography, provides enantiomerically pure stereoisomers 8 and 9 (step a). Removal of the protective group in intermediates 8 and 9, applying methods known in the art (e.g., a Boc group using TFA in DCM or HCl in dioxane or diethylether at temperatures between 0° C. and room temperature, a Cbz group using hydrogen in the presence of a suitable catalyst such as Pd or Pd(OH)₂ on charcoal in a suitable solvent such as MeOH, EtOH, ethyl acetate or mixtures therefore and as described for example in “Protective Groups in Organic Chemistry” by T. W. Greene and P. G. M. Wuts, 4th Ed., 2006, Wiley, New York) provides pure trans-configured intermediates 1B and 1C.

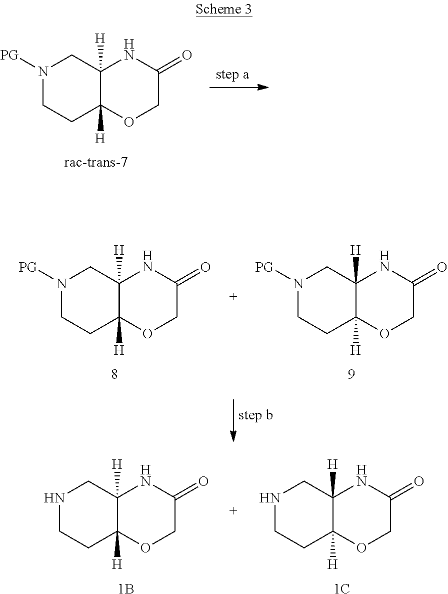

In some embodiments, intermediates 2 are intermediates of type II in which m, and n are as described herein, B is an optionally further substituted aryl or heteroaryl ring and R¹ to R³ are each independently selected from hydrogen, substituted or unsubstituted (cyclo)alkyl, (cyclo)alkoxy, substituted or unsubstituted aryl, R^{b}R^{c}N, cyano, heterocycle, methylsulfonyl and halogen, wherein substituted alkyl, aryl and heteroaryl is as defined herein, R^{b} is hydrogen, alkyl or aryl and R^{c} is alkyl or aryl or R^{b} and R^{c}, taken together with the nitrogen atom to which they are attached, form an optionally further substituted 4-11-membered, mono- or bicyclic heterocyclic ring. Intermediates of that type can be prepared by methods well known in the art and as exemplified by the general synthetic procedures outlined in Scheme 4.

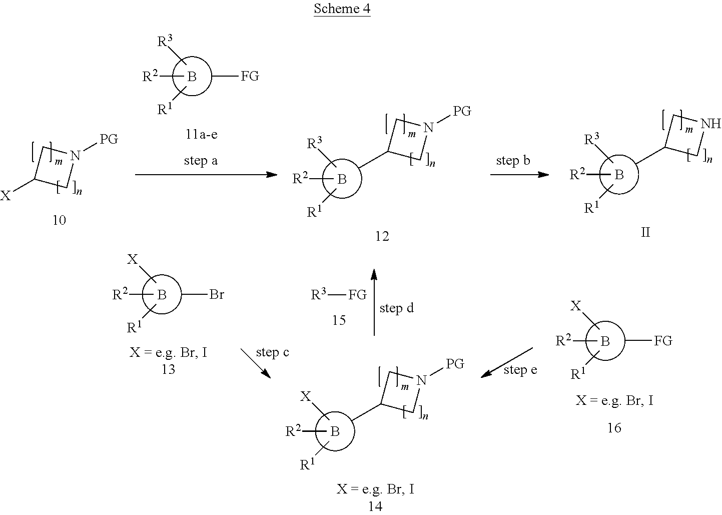

Commercially available intermediates 10 in which PG signifies a suitable protecting group and X is bromide or iodide can be subjected to cross-coupling reactions such as Negishi, Heck, Stille, Suzuki, Sonogashira or Buchwald-Hartwig coupling reactions with compounds 11, either commercially available or prepared by methods known in the art, in which FG signifies a suitable functional group such as, e.g., chloro, bromo, iodo, —OSO₂alkyl (e.g., mesylate (methanesulfonate)), —OSO₂fluoroalkyl (e.g., triflate (trifluoromethanesulfonate)) or —OSO₂aryl (e.g., tosylate (p-toluenesulfonate)) (step a). Reactions of this type are broadly described in literature and well known to persons skilled in the art.

For example, intermediates 10 can be reacted with aryl or heteroaryl boronic acids 11a (FG=B(OH)₂) or boronic esters 11b (FG=e.g., 4,4,5,5-tetramethyl-2-phenyl-1,3,2-dioxaborolane (pinacol) ester) either commercially available or prepared using literature procedures as described for example in “Boronic Acids—Preparation and Applications in Organic Synthesis and Medicine” by Dennis G. Hall (ed.), 1^{st} Ed., 2005, John Wiley & Sons, New York, using a suitable catalyst (e.g., dichloro[1,1′-bis(diphenylphosphino)-ferrocene]palladium(II) dichloromethane adduct, tetrakis(triphenylphosphine)palladium(0) or palladium(II)acetate with triphenylphosphine) in an appropriate solvent (e.g., dioxane, dimethoxyethane, water, toluene, DMF or mixtures thereof) and a suitable base (e.g., Na₂CO₃, NaHCO₃, KF, K₂CO₃ or TEA) at temperatures between room temperature and the boiling point of the solvent or solvent mixture, to yield intermediates 12 (step a). Suzuki reactions of this type are broadly described in literature (e.g., Suzuki, A., Pure Appl. Chem. 1991, 63, 419; Suzuki, A., Miyaura, N., Chem. Rev. 1995, 95, 2457; Suzuki, A., J Organomet. Chem. 1999, 576, 147; Polshettiwar, N., Decottignies, A., Len, C., Fihri, A., Chem Sus Chem 2010, 3, 502) and are well known to those skilled in the art. Alternatively, aryl- or heteroaryl-trifluoroborates 11c (FG=BF₃) can be used in the cross-coupling reaction applying a palladium catalyst such as, e.g., tetrakis(triphenylphosphine)-palladium(0), palladium(II) acetate or dichloro[1,1′-bis(diphenylphosphino)ferrocene]-palladium(II) dichloromethane adduct in the presence of a suitable base such as cesium carbonate or potassium phosphate in solvents such as toluene, THF, dioxane, water or mixtures thereof, at temperatures between room temperature and the boiling point of the solvent or solvent mixture.

Alternatively, intermediates 10 can be reacted with aryl or heteroaryl stannanes 11d in which FG is Sn(alkyl)₃ and alkyl is preferable n-butyl or methyl, using a suitable catalyst and solvent such as, e.g. tetrakis(triphenylphosphine)-palladium(0) in DMF at temperatures between room temperature and the boiling point of the solvent or solvent mixture to provide intermediates 12 (step a). Stille reactions of that type are well known in the art and described in literature, e.g., Farina, V., Krishnamurthy, V., Scott, W. J., Org. React. 1997, 50, 1-652; Cordovilla, C., Bartolomé, C., Martinez-Ilarduya, J. M., Espinet, P., ACS Catal. 2015, 5, 3040.

Furthermore, intermediates 10 can be reacted with aryl or heteroarylzinc halides 11e in which FG is ZnHal and Hal preferably bromide or iodide, either commercially available or prepared by literature methods, using an appropriate catalyst and solvent system such as, e.g., [1,1′-bis(diphenylphosphino)ferrocene]dichloropalladium(II) and copper(I)iodide in DMA, or tetrakis(triphenylphosphine)palladium(0) in THF or DMF at temperatures between room temperature and the boiling point of the solvent to provide intermediates 12 (step a). Negishi reactions of that type are well known in the art and also described in literature, e.g., Gayryushin, A., Kofink, C., Manolikakes, G., Knochel, P., Org. Lett. 2005, 7, 4871; Haas, D., Hammann, J. M., Greiner, R., Knochel, P., ACS Catal. 2016, 6, 1540; Negishi, E.-I., Acc. Chem. Res. 1982, 15, 340.

Alternatively, intermediates 12 may be prepared by converting intermediates 10 in which X is for example iodide into the corresponding zinc species by applying literature methods (e.g., reaction of 10 with Zn powder in the presence of chlorotrimethylsilane and 1,2-dibromoethane in a suitable solvent such as DMA) and coupling of the zinc species with aryl- or heteroarylbromides- or iodides under the conditions mentioned before.

Alternatively, intermediates 10 in which X is preferably bromide can be subjected to a cross-electrophile coupling with aryl- or heteroarylbromides 11f in which FG signifies bromide under irradiation with a 420 nm blue light lamp using an appropriate photo catalyst such as bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (Ir[dF(CF₃)ppy]₂(dtbbpy))PF₆), a nickel catalyst like NiCl₂ glyme (dichloro(dimethoxyethane)nickel), 4,4′-di-tert-butyl-2,2′-dipyridyl and tris(trimethylsilyl)silane, in the presence of a suitable base such as anhydrous sodium carbonate in a solvent like DME. Reactions of this type are described in literature, e.g. Zhang, P., Le, C., MacMillan, D. W. C., J. Am. Chem. Soc. 2016, 138, 8084 (step a).

Removal of the protective group from intermediates 12 applying methods well known in the art and as described for example under Scheme 2 (step c), furnishes intermediates II (step b).

Intermediates 12 may alternatively be prepared from intermediates 10 and aryl or heteroaryl bromides 13, either commercially available or prepared by methods known in the art, applying the transformations described before under step a to furnish intermediates 14 (step c).

Intermediates 14 may alternatively be prepared from intermediates 10 and aryl or heteroaryl boronic acids 16a (FG=B(OH)₂) or boronic esters 16b (FG=e.g., 4,4,5,5-tetramethyl-2-phenyl-1,3,2-dioxaborolane (pinacol) ester), either commercially available or prepared by methods known in the art, by a nickel-mediated alkyl-aryl Suzuki coupling reaction well known in the art and also described in literature, e.g., Duncton, M. A. J., Estiarte, M. A., Tan, D., Kaub, C., O'Mahony, D. J. R., Johnson, R. J., Cox, M., Edwards, W. T., Wan, M., Kincaid, J., Kelly, M. G., Org. Lett. 2008, 10, 3259; González-Bobes, F., Fu, G. C., J Am. Chem. Soc. 2006, 128, 5360 (step e).

Intermediates 14 can be further reacted with compounds 15 applying the same synthetic strategies as described before under step a to provide intermediates 12 (step d).

Intermediates 12 in which R³ signifies an amine group of type R^{b}R^{c}N in which R^{b} is hydrogen, alkyl or aryl and R^{c} is alkyl or aryl or in which R^{b} and R^{c}, taken together with the nitrogen atom to which they are attached, form an optionally further substituted 4-11-membered, mono- or bicyclic heterocyclic ring, can be synthesized for example from reaction of 14 with primary or secondary amines R^{b}R^{c}NH and using for example a suitable catalyst (e.g., Pd(OAc)₂, Pd₂(dba)₃), ligand (e.g., BINAP, Xphos, BrettPhos, RuPhos), base (e.g., Cs₂CO₃, K₂CO₃, KOt-Bu, LiHMDS, K₃PO₄) and solvent (e.g., toluene, THF, dioxane)). Buchwald-Hartwig reactions of that type are known in the art and described in literature (e.g., Surry, D. S., Buchwald, S. L., Angew. Chem. Int. Ed. 2008, 47, 6338; Evano, G., Blanchard, N., Toumi, M., Chem. Rev. 2008, 108, 3054; Heravi, M. M., Kheilkordi, Z., Zadsirjan, V., Heydari, M., Malmir, M., J Organomet. Chem. 2018, 861, 17) (step d).

Intermediates of type III in which R^{L} is as defined herein, can be prepared by a variety of conditions, which may be exemplified by the general synthetic procedure outlined in Scheme 5.

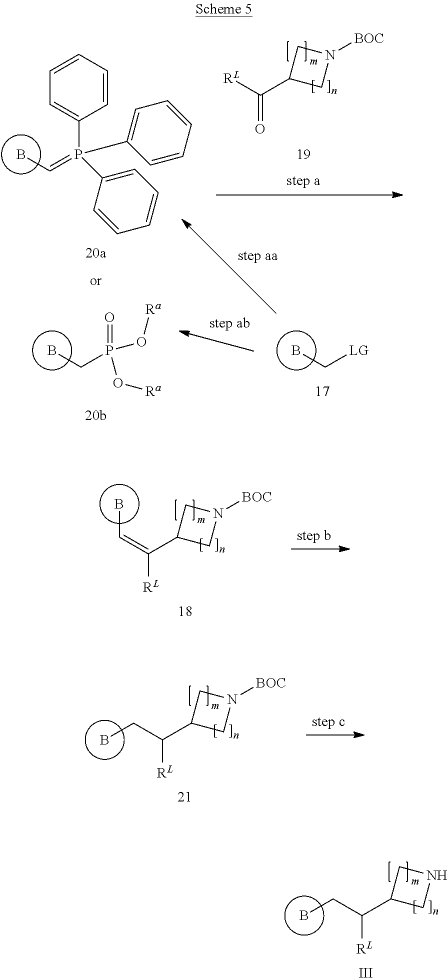

Intermediates 18 can be prepared by an olefination reaction such as the widely described Wittig or Horner-Wadsworth-Emmons (HWE) reaction using phosphonium salts or phosphonate carbanions 20a or 20b with aldehydes or ketones 19, which are either commercially available or prepared by methods known in the art.

Wittig reaction with alkylidene triphenylphosphoranes of type 20a in a suitable solvent such as, e.g., THF, Methyl-THF or DMSO provide intermediates 18 (step a). Phosphoranes 20a can be formed by treating the corresponding phosphonium salts with a suitable base such as BuLi, NaH, or KOtBu in a suitable solvent such as THF, dioxane or Methyl-THF and may be isolated or used in situ. Phosphonium salts in turn are readily available from an aryl halide 17, wherein LG is a halogen selected from Cl, Br or I and B is as defined herein, and triphenylphosphine in a suitable solvent such as toluene (step aa). Heating may be applied to accelerate the reaction or drive the reaction to completion (e.g., H. J. Cristau, F. Plenat in PATAIS Chemistry of Functional Groups, Frank R. Hartley (ed.), 7 Aug. 2006, Saul Patai (series ed.)).

Alternatively, intermediates 18 can be obtained using a Horner-Wadsworth-Emmons (HWE) reaction using aldehydes/ketones 19 and phosphonates 20b, wherein R^{a} is alkyl, for example methyl or ethyl. Phosphonates 20b are in situ α-metalated using a suitable base and solvent such as NaH, nBuLi or KOtBu in THF (step a). Phosphonates 20b are readily prepared using for example the Arbuzov reaction by alkylation of an aryl halide 17 wherein LG is a halogen selected from Cl, Br or I and B is as defined herein, with commercially available trialkyl phosphite (step ab, see e.g., Brill, T. B., Landon, S. J., Chem. Rev. 1984, 84, 577).

Olefination reactions of both types are broadly described in literature (e.g., Maryanoff, B. E., Reitz, A. B., Chem. Rev. 1989, 89, 863; Boutagy, J., Thomas, R., Chem. Rev. 1974, 74, 87; Bisceglia, J. A., Orelli, L. R., Current Org. Chem. 2015, 19, 744; Wadsworth Jr., W. S., Org. React. 1977, 25, 73; Nicolaou, K. C., Harter, M. W., Gunzner, J. L., Nadin, A., Liebigs Ann./Recueil 1997, 1283; Stec, W. J., Acc. Chem. Res. 1983, 16, 411) (step a).

The double bond in intermediates 18 can be reduced for example by hydrogenation under atmospheric pressure in the presence of a suitable catalyst such as Pd(OH)₂ or Pd/C in a suitable solvent such as MeOH, EtOH or EtOAc or mixtures thereof to yield intermediates 21 (step b).

Removal of the protective group from intermediates 21 applying methods well known in the art and as described for example under Scheme 2 (step c), furnishes intermediates III (step c).

Intermediates of type IV, can be prepared by a variety of conditions, which may be exemplified by the general synthetic procedure outlined in Scheme 6.

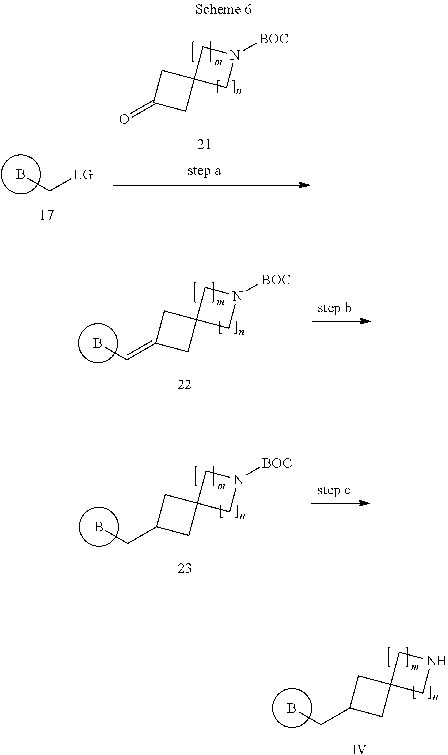

Starting from aryl or heterobenzyl halides 17, wherein LG is selected from Cl, Br or I and B is as defined herein, intermediates 22 can be prepared by an olefination reaction such as the widely described Wittig or Homer-Wadsworth-Emmons (HWE) reaction using phosphonium salts or phosphonate carbanions with spiro ketones 21, which are either commercially available or prepared by methods known in the art, as described above (step a).

The double bond in intermediates 22 can be reduced for example by hydrogenation under atmospheric pressure in the presence of a suitable catalyst such as Pd(OH)₂ or Pd/C in a suitable solvent such as MeOH, EtOH or EtOAc or mixtures thereof to yield intermediates 23 (step b).

Removal of the protective group from intermediates 23 applying methods well known in the art and as described for example under Scheme 2 (step c), furnishes intermediates IV (step c).

Intermediates of type V in which R^{L} is as defined herein, can be prepared by a variety of conditions, which may be exemplified by the general synthetic procedure outlined in Scheme 7.

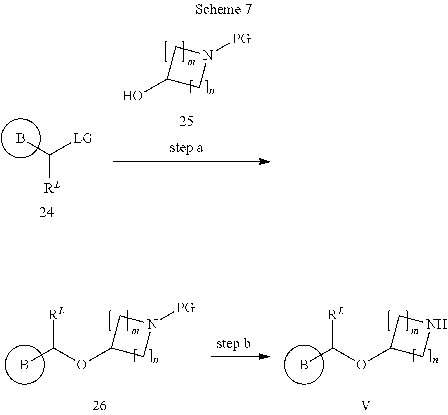

Intermediates 26 may be prepared from alcohols 25 in which PG is a suitable protective group such as a Cbz, Boc or Bn that can be alkylated with compounds 24 in which LG is a suitable leaving group such as chlorine, bromine, iodine, OSO₂alkyl (e.g., methanesulfonate), OSO₂fluoroalkyl (e.g., trifluoromethanesulfonate) or OSO₂aryl (e.g., p-toluenesulfonate) using a suitable base, such as sodium hydride, KOtBu, in an appropriate solvent (e.g., in DMF or THF) at temperatures between 0° C. and the boiling temperature of the solvent (step a).

Removal of the protective group from intermediates 23 applying methods well known in the art and as described for example under Scheme 2 (step c), furnishes intermediates V (step b).

In one aspect, the present invention provides a process of manufacturing the urea compounds of formula (Ia) or (Ib) described herein, comprising:
reacting a first amine 4a,5,6,7,8,8a-hexahydro-4H-pyrido[4,3-b][1,4]oxazin-3-one (1)

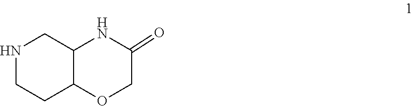 [SMILES: O=C1COC2CCNCC2N1]

with a second amine of formula 2, wherein A, L, and B are as described herein

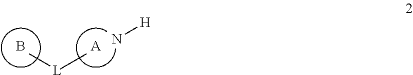

in the presence of a base and a urea forming reagent,
   to form said compound of formula (Ia) or (Ib).

In one embodiment, there is provided a process according to the invention, wherein said base is sodium bicarbonate.

In one embodiment, there is provided a process according to the invention, wherein said urea forming reagent is selected from bis(trichloromethyl) carbonate, phosgene, trichloromethyl chloroformate, (4-nitrophenyl)carbonate and 1,1′-carbonyldiimidazole, preferably wherein said urea forming reagent is bis(trichloromethyl) carbonate.

In one aspect, the present invention provides a compound of formula (Ia) or (Ib) as described herein, when manufactured according to any one of the processes described herein.

### MAGL Inhibitory Activity

Compounds of the present invention are MAGL inhibitors. Thus, in one aspect, the present invention provides the use of compounds of formula (Ia) or (Ib) as described herein for inhibiting MAGL in a mammal.

In a further aspect, the present invention provides compounds of formula (Ia) or (Ib) as described herein for use in a method of inhibiting MAGL in a mammal.

In a further aspect, the present invention provides the use of compounds of formula (Ia) or (Ib) as described herein for the preparation of a medicament for inhibiting MAGL in a mammal.

In a further aspect, the present invention provides a method for inhibiting MAGL in a mammal, which method comprises administering an effective amount of a compound of formula (Ia) or (Ib) as described herein to the mammal.

Compounds were profiled for MAGL inhibitory activity by determining the enzymatic activity by following the hydrolysis of the natural substrate 2-arachidonoylglycerol resulting in arachidonic acid, which can be followed by mass spectrometry. This assay is hereinafter abbreviated “2-AG assay”.

The 2-AG assay was carried out in 384 well assay plates (PP, Greiner Cat #784201) in a total volume of 20 μL. Compound dilutions were made in 100% DMSO (VWR Chemicals 23500.297) in a polypropylene plate in 3-fold dilution steps to give a final concentration range in the assay from 12.5 μM to 0.8 μM. 0.25 μL compound dilutions (100% DMSO) were added to 9 μL MAGL in assay buffer (50 mM TRIS (GIBCO, 15567-027), 1 mM EDTA (Fluka, 03690-100 ml) and 0.01% (v/v) Tween. After shaking, the plate was incubated for 15 min at rt. To start the reaction, 10 μL 2-arachidonoylglycerol in assay buffer was added. The final concentrations in the assay was 50 μM MAGL and 8 μM 2-arachidonoylglyerol. After shaking and 30 min incubation at rt, the reaction was quenched by the addition of 40 μL of ACN containing 4 μM of d8-arachidonic acid. The amount of arachidonic acid was traced by an online SPE system (Agilent Rapidfire) coupled to a triple quadrupole mass spectrometer (Agilent 6460). A C18 SPE cartridge (G9205A) was used in an ACN/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | IC₅₀ MAGL |
|---|---|
| Example | [nM\| |
| 1 | 235 |
| 2 | 39 |
| 3 | 95 |
| 4 | 15 |
| 5 | 327 |
| 6 | 143 |
| 7 | 424 |
| 8 | 56 |
| 9 | 85 |
| 10 | 43 |
| 11 | 47 |
| 12 | 7 |
| 13 | 93 |
| 14 | 28 |
| 15 | 357 |
| 16 | 58 |
| 17 | 24 |
| 18 | 12 |
| 19 | 32 |
| 20 | 220 |
| 21 | 78 |
| 22 | 920 |
| 23 | 411 |
| 24 | 208 |
| 25 | 5 |
| 26 | 118 |
| 27 | 7 |
| 28 | 2558 |
| 29 | 1305 |
| 30 | 530 |
| 31 | 2010 |

In one aspect, the present invention provides compounds of formula (Ia) or (Ib) and their pharmaceutically acceptable salts as described herein, wherein said compounds of formula (Ia) or (Ib) and their pharmaceutically acceptable salts have IC₅₀'s for MAGL inhibition below 25 μM, preferably below 10 μM, more preferably below 5 μM as measured in the MAGL assay described herein.

In one embodiment, compounds of formula (Ia) or (Ib) and their pharmaceutically acceptable salts as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

### Using the Compounds of the Invention

In one aspect, the present invention provides compounds of formula (I) as described herein for use as therapeutically active substance.

In a further aspect, the present invention provides the use of compounds of formula (I) as described herein for the treatment or prophylaxis of neuroinflammation, neurodegenerative diseases, pain, cancer, mental disorders and/or inflammatory bowel disease in a mammal.

In one embodiment, the present invention provides the use of compounds of formula (I) as described herein for the treatment or prophylaxis of neuroinflammation and/or neurodegenerative diseases in a mammal.

In one embodiment, the present invention provides the use of compounds of formula (I) as described herein for the treatment or prophylaxis of neurodegenerative diseases in a mammal.

In one embodiment, the present invention provides the use of compounds of formula (I) as described herein for the treatment or prophylaxis of cancer in a mammal.

In one embodiment, the present invention provides the use of compounds of formula (I) as described herein for the treatment or prophylaxis of inflammatory bowel disease in a mammal.

In one embodiment, the present invention provides the use of compounds of formula (I) as described herein for the treatment or prophylaxis of pain in a mammal.

In one aspect, the present invention provides the use of compounds of formula (I) as described herein for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I) as described herein for the treatment or prophylaxis of multiple sclerosis, Alzheimer's disease and/or Parkinson's disease in a mammal.

In a particularly preferred embodiment, the present invention provides the use of compounds of formula (I) as described herein for the treatment or prophylaxis of multiple sclerosis in a mammal.

In one aspect, the present invention provides compounds of formula (I) as described herein for use in the treatment or prophylaxis of neuroinflammation, neurodegenerative diseases, pain, cancer, mental disorders and/or inflammatory bowel disease in a mammal.

In one embodiment, the present invention provides compounds of formula (I) as described herein for use in the treatment or prophylaxis of neuroinflammation and/or neurodegenerative diseases in a mammal.

In one embodiment, the present invention provides compounds of formula (I) as described herein for use in the treatment or prophylaxis of cancer in a mammal.

In one embodiment, the present invention provides compounds of formula (I) as described herein for use in the treatment or prophylaxis of neurodegenerative diseases in a mammal.

In one embodiment, the present invention provides compounds of formula (I) as described herein for use in the treatment or prophylaxis of inflammatory bowel disease in a mammal.

In one embodiment, the present invention provides compounds of formula (I) as described herein for use in the treatment or prophylaxis of pain in a mammal.

In one aspect, the present invention provides compounds of formula (I) as described herein for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides compounds of formula (I) as described herein for use in the treatment or prophylaxis of multiple sclerosis, Alzheimer's disease and/or Parkinson's disease in a mammal.

In a particularly preferred embodiment, the present invention provides compounds of formula (I) as described herein for use in the treatment or prophylaxis of multiple sclerosis in a mammal.

In one aspect, the present invention provides the use of compounds of formula (I) as described herein for the preparation of a medicament for the treatment or prophylaxis of neuroinflammation, neurodegenerative diseases, pain, cancer, mental disorders and/or inflammatory bowel disease in a mammal.

In one embodiment, the present invention provides the use of compounds of formula (I) as described herein for the preparation of a medicament for the treatment or prophylaxis of neuroinflammation and/or neurodegenerative diseases in a mammal.

In one embodiment, the present invention provides the use of compounds of formula (I) as described herein for the preparation of a medicament for the treatment or prophylaxis of neurodegenerative diseases in a mammal.

In one embodiment, the present invention provides the use of compounds of formula (I) as described herein for the preparation of a medicament for the treatment or prophylaxis of cancer in a mammal.

In one embodiment, the present invention provides the use of compounds of formula (I) as described herein for the preparation of a medicament for the treatment or prophylaxis of inflammatory bowel disease in a mammal.

In one embodiment, the present invention provides the use of compounds of formula (I) as described herein for the preparation of a medicament for the treatment or prophylaxis of pain in a mammal.

In a further aspect, the present invention provides the use of compounds of formula (I) as described herein for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

In a preferred embodiment, the present invention provides the use of compounds of formula (I) as described herein for the preparation of a medicament for the treatment or prophylaxis of multiple sclerosis, Alzheimer's disease and/or Parkinson's disease in a mammal.

In a particularly preferred embodiment, the present invention provides the use of compounds of formula (I) as described herein for the preparation of a medicament for the treatment or prophylaxis of multiple sclerosis in a mammal.

In one aspect, the present invention provides a method for the treatment or prophylaxis of neuroinflammation, neurodegenerative diseases, pain, cancer, mental disorders and/or inflammatory bowel disease in a mammal, which method comprises administering an effective amount of a compound of formula (I) as described herein to the mammal.

In one embodiment, the present invention provides a method for the treatment or prophylaxis of neuroinflammation and/or neurodegenerative diseases in a mammal, which method comprises administering an effective amount of a compound of formula (I) as described herein to the mammal.

In one embodiment, the present invention provides a method for the treatment or prophylaxis of neurodegenerative diseases in a mammal, which method comprises administering an effective amount of a compound of formula (I) as described herein to the mammal.

In one embodiment, the present invention provides a method for the treatment or prophylaxis of cancer in a mammal, which method comprises administering an effective amount of a compound of formula (I) as described herein to the mammal.

In one embodiment, the present invention provides a method for the treatment or prophylaxis of inflammatory bowel disease in a mammal, which method comprises administering an effective amount of a compound of formula (I) as described herein to the mammal.

In one embodiment, the present invention provides a method for the treatment or prophylaxis of pain in a mammal, which method comprises administering an effective amount of a compound of formula (I) as described herein to the mammal.

In a further aspect, the present invention provides a method for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal, which method comprises administering an effective amount of a compound of formula (I) as described herein to the mammal.

In a preferred embodiment, the present invention provides a method for the treatment or prophylaxis of multiple sclerosis, Alzheimer's disease and/or Parkinson's disease in a mammal, which method comprises administering an effective amount of a compound of formula (I) as described herein to the mammal.

In a particularly preferred embodiment, the present invention provides a method for the treatment or prophylaxis of multiple sclerosis in a mammal, which method comprises administering an effective amount of a compound of formula (I) as described herein to the mammal.

### Pharmaceutical Compositions and Administration

In one aspect, the present invention provides a pharmaceutical composition comprising a compound of formula (Ia) or (Ib) as described herein and a therapeutically inert carrier.

In one embodiment, the present invention provides the pharmaceutical compositions disclosed in examples 32 and 33, respectively.

The compounds of formula (Ia) or (Ib) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (Ia) or (Ib) and their pharmaceutically acceptable salts can be processed with pharmaceutically inert, inorganic or organic adjuvants for the production of tablets, coated tablets, dragées and hard gelatin capsules. Lactose, corn starch or derivatives thereof, talc, stearic acid or its salts etc. can be used, for example, as such adjuvants for tablets, dragées and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1 and Example 2

### (+)- or (−)-trans-6-[3-(4-tert-Butylphenyl)azetidine-1-carbonyl]-4,4a,5,7,8,8a-hexahydropyrido[4,3-b][1,4]oxazin-3-one and

### (−)- or (+)-trans-6-[3-(4-tert-Butylphenyl)azetidine-1-carbonyl]-4,4a,5,7,8,8a-hexahydropyrido[4,3-b][1,4]oxazin-3-one

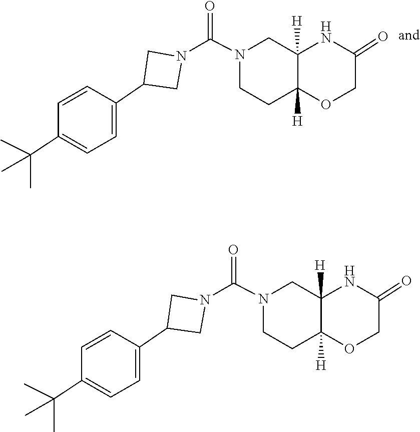 [SMILES: CC(C)(C)c1ccc(C2CN(C(=O)N3CC[C@H]4OCC(=O)N[C@@H]4C3)C2)cc1 ; CC(C)(C)c1ccc(C2CN(C(=O)N3CC[C@@H]4OCC(=O)N[C@H]4C3)C2)cc1]

To a solution of rac-trans-hexahydro-2H-pyrido[4,3-b][1,4]oxazin-3(4H)-one hydrochloride (49.8 mg, 233 μmol, 1.0 equiv; BB 1) and trimethylamine (145 mg, 200 μL, 1.43 mmol, 6.2 equiv) in acetonitrile (1.0 mL) was added 1,1′-carbonyl-di(1,2,4-triazole) (38.2 mg, 233 μL, 1.0 equiv) and the reaction mixture stirred at rt. After 1 h, 3-(4-(tert-butyl)phenyl)azetidine 4-methylbenzenesulfonate (84.1 mg, 233 μmol, equiv 1.0; BB 2) was added and stirring continued at 50° C. for 1 h. The reaction mixture was concentrated and the residue was purified by preparative HPLC to give the desired product as a white solid (42.8 mg, 50%). The enantiomers were separated by chiral SFC (Chiralpak AD-H column, 220 nm, 5 μm, 250×20 mm) to yield Example 1 (11.0 mg, 13%; first eluting isomer) and Example 2 (11.0 mg, 13%; second eluting isomer) as white solids. MS (ESI): m/z=372.3 [M+H]⁺ for both examples.

### Example 3 and Example 4

### (+)- or (−)-trans-6-[3-[4-[1-(Trifluoromethyl)cyclopropyl]phenyl]azetidine-1-carbonyl]-4,4a,5,7,8,8a-hexahydropyrido[4,3-b][1,4]oxazin-3-one and

### (−)- or (+)-trans-6-[3-[4-[1-(Trifluoromethyl)cyclopropyl]phenyl]azetidine-1-carbonyl]-4,4a,5,7,8,8a-hexahydropyrido[4,3-b][1,4]oxazin-3-one

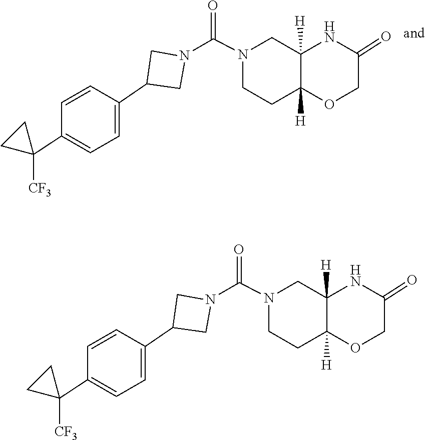 [SMILES: O=C1CO[C@@H]2CCN(C(=O)N3CC(c4ccc(C5(C(F)(F)F)CC5)cc4)C3)C[C@H]2N1 ; O=C1CO[C@H]2CCN(C(=O)N3CC(c4ccc(C5(C(F)(F)F)CC5)cc4)C3)C[C@@H]2N1]

To a solution of rac-trans-hexahydro-2H-pyrido[4,3-b][1,4]oxazin-3(4H)-one hydrochloride (49.8 mg, 233 μmol, 1.0 equiv; BB 1) and trimethylamine (145 mg, 200 μL, 1.43 mmol, 6.2 equiv) in acetonitrile (1.0 mL) was added 1,1′-carbonyl-di(1,2,4-triazole) (38.2 mg, 233 μL, 1.0 equiv) and the reaction mixture stirred at rt. After 1 h, 3-[4-[1-(trifluoromethyl)cyclopropyl]phenyl]azetidine 4-methylbenzenesulfonate (96.3 mg, 233 μmol, equiv 1.0; BB 3) was added and stirring continued at 50° C. for 1 h. The reaction mixture was concentrated and the residue was purified by preparative HPLC to give the desired product as a white solid (60.6 mg, 55%). The enantiomers were separated by chiral SFC (Chiralpak AD-H column, 220 nm, 5 μm, 250×20 mm) to yield Example 3 (12.9 mg, 23%; first eluting isomer) and Example 4 (12.1 mg, 22%; second eluting isomer) as white solids. MS (ESI): m/z=424.4 [M+H]⁺ for both examples.

### Example 5 and Example 6

### (+)- or (−)-trans-6-[3-[[2-Fluoro-4-(trifluoromethyl)phenyl]methoxy]azetidine-1-carbonyl]-4,4a,5,7,8,8a-hexahydropyrido[4,3-b][1,4]oxazin-3-one and

### (−)- or (+)-trans-6-[3-[[2-Fluoro-4-(trifluoromethyl)phenyl]methoxy]azetidine-1-carbonyl]-4,4a,5,7,8,8a-hexahydropyrido[4,3-b][1,4]oxazin-3-one

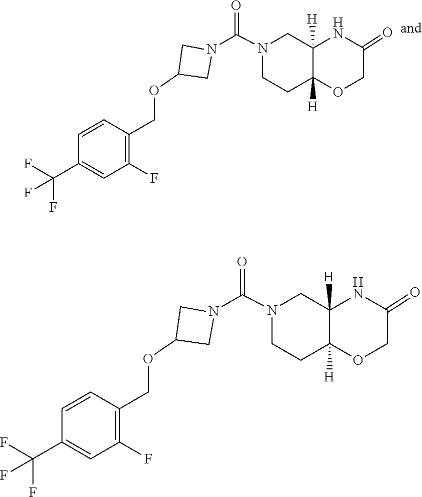 [SMILES: O=C1CO[C@@H]2CCN(C(=O)N3CC(OCc4ccc(C(F)(F)F)cc4F)C3)C[C@H]2N1 ; O=C1CO[C@H]2CCN(C(=O)N3CC(OCc4ccc(C(F)(F)F)cc4F)C3)C[C@@H]2N1]

To an ice-cold solution of bis(trichloromethyl) carbonate (97 mg, 0.33 mmol, 0.7 equiv) in DCM (4 mL) were added sodium bicarbonate (157 mg, 1.87 mmol, 4.0 equiv) and 3-[[2-fluoro-4-(trifluoromethyl)phenyl]methoxy]azetidine 4-methylbenzenesulfonic acid (236 mg, 561 μmol, 1.2 equiv; BB 4) and the reaction mixture stirred at rt. After 8 h, rac-trans-hexahydro-2H-pyrido[4,3-b][1,4]oxazin-3(4H)-one hydrochloride (90 mg, 467 μmol, 1.0 equiv; BB 1) and DIPEA (242 mg, 326 μL, 1.87 mmol, 4.0 equiv) were added and stirring continued at rt for 5 h. The reaction mixture was poured on water and DCM and the layers were separated. The aqueous layer was extracted twice with DCM. The organic layers were washed twice with water, dried over MgSO₄, filtered and evaporated. The crude product was purified by preparative HPLC to give the desired product as a colorless solid (86 mg, 42%). The enantiomers were separated by chiral SFC (Chiralpak AD-H column, 220 nm, 5 μm, 250×20 mm) to yield Example 5 (41 mg, 51%; first eluting isomer) and Example 6 (36 mg, 45%; second eluting isomer) as light brown solids. MS (ESI): m/z=432.3 [M+H]⁺ for Example 5 and MS (ESI): m/z=432.2 [M+H]⁺ for Example 6.

### Example 7

### (+)- or (−)-trans-6-[3-[3-Chloro-4-(trifluoromethoxy)phenyl]azetidine-1-carbonyl]-4,4a,5,7,8,8a-hexahydropyrido[4,3-b][1,4]oxazin-3-one

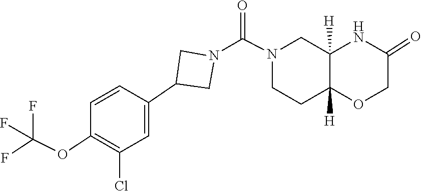 [SMILES: O=C1CO[C@@H]2CCN(C(=O)N3CC(c4ccc(OC(F)(F)F)c(Cl)c4)C3)C[C@H]2N1]

To a solution of (+)-trans-hexahydro-2H-pyrido[4,3-b][1,4]oxazin-3(4H)-one hydrochloride (17.3 mg, 90 μmol, 1.0 equiv; BB 5A) and trimethylamine (64.2 mg, 89 μL, 630 μmol, 7.0 equiv) in acetonitrile (1.0 mL) was added 1,1′-carbonyl-di(1,2,4-triazole) (14.8 mg, 90 μmol, 1.0 equiv) and the reaction mixture stirred at rt. After 1 h, 3-[3-chloro-4-(trifluoromethoxy)phenyl]azetidine 2,2,2-trifluoroacetic acid (39.5 mg, 108 μmol, equiv 1.2; CAS RN 1260891-17-5) was added and stirring continued at 60° C. for 1 h. The reaction mixture was concentrated and the residue was purified by preparative HPLC to give the desired product as an off-white solid (3.4 mg, 9%). MS (ESI): m/z=434.3 [M+H]⁺.

### Example 8

### (−)- or (+)-trans-6-[3-[3-Chloro-4-(trifluoromethoxy)phenyl]azetidine-1-carbonyl]-4,4a,5,7,8,8a-hexahydropyrido[4,3-b][1,4]oxazin-3-one

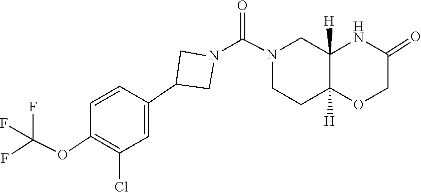 [SMILES: O=C1CO[C@H]2CCN(C(=O)N3CC(c4ccc(OC(F)(F)F)c(Cl)c4)C3)C[C@@H]2N1]

To a solution of (−)-trans-hexahydro-2H-pyrido[4,3-b][1,4]oxazin-3(4H)-one hydrochloride (17.3 mg, 90 μmol, 1.0 equiv; BB 5B) and trimethylamine (64.2 mg, 89 μL, 630 μmol, 7.0 equiv) in acetonitrile (1.0 mL) was added 1,1′-carbonyl-di(1,2,4-triazole) (14.8 mg, 90 μmol, 1.0 equiv) and the reaction mixture stirred at rt. After 1 h, 3-[3-chloro-4-(trifluoromethoxy)phenyl]azetidine 2,2,2-trifluoroacetic acid (39.5 mg, 108 μmol, equiv 1.2; CAS RN 1260891-17-5) was added and stirring continued at 60° C. for 1 h. The reaction mixture was concentrated and the residue was purified by preparative HPLC to give the desired product as an off-white solid (2.6 mg, 7%). MS (ESI): m/z=434.3 [M+H]⁺.

If not indicated otherwise the following examples were synthesized in analogy to the synthesis described for Example 7 and Example 8 using suitable building blocks, respectively.

| | | | | MS |
|---|---|---|---|---|
| Ex. | Systematic Name | Structure | Building blocks | m/z |
| 9 | (+)- or (−)-trans-6[3- [2-[2-Fluoro-4- (trifluoromethyl) phenyl] ethyl]azetidine-1- carbonyl]- 4,4a,5,7,8,8a- hexahydropyrido[4,3- b][1,4]oxazin-3-one | 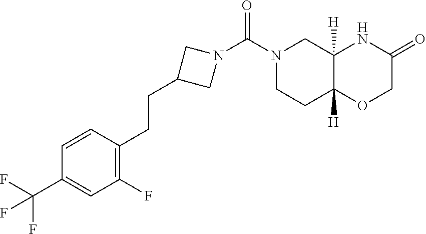 [SMILES: O=C1CO[C@@H]2CCN(C(=O)N3CC(CCc4ccc(C(F)(F)F)cc4F)C3)C[C@H]2N1] | (+)-trans- 4a,5,6,7,8,8a- Hexahydro-4H- pyrido[4,3- b][1,4]oxazin-3-one hydrochloride (BB 5A) and 3-[2-[2- Fluoro-4- (trifluoromethyl) phenyl] ethyl]azetidine 4- methylbenzenesulfonic acid (BB 6) | 430.3 [M + H]⁺ |
| 10 | (−)- or (+)-trans-6-[3- [2-[2-Fluoro-4- (trifluoromethyl) phenyl] ethyl]azetidine-1- carbonyl]- 4,4a,5,7,8,8a- hexahydropyrido[4,3- b][1,4]oxazin-3-one | 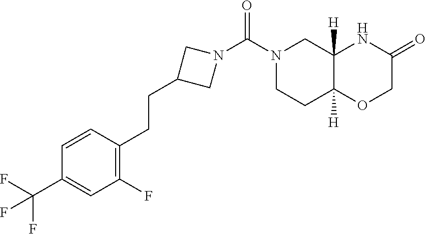 [SMILES: O=C1CO[C@H]2CCN(C(=O)N3CC(CCc4ccc(C(F)(F)F)cc4F)C3)C[C@@H]2N1] | (−)-trans- 4a,5,6,7,8,8a- Hexahydro-4H- pyrido[4,3- b][1,4]oxazin-3-one hydrochloride (BB 5B) and 3-[2-[2- Fluoro-4- (trifluoromethyl) phenyl] ethyl]azetidine 4- methylbenzenesulfonic acid (BB 6) | 430.3 [M + H]⁺ |
| 11 | (+)- or (−)-trans-6-[3- (4-Phenoxyphenyl) azetidine- 1-carbonyl]- 4,4a,5,7,8,8a- hexahydropyrido[4,3- b][1,4]oxazin-3-one | 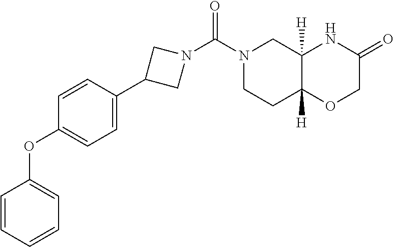 [SMILES: O=C1CO[C@@H]2CCN(C(=O)N3CC(c4ccc(Oc5ccccc5)cc4)C3)C[C@H]2N1] | (+)-trans- 4a,5,6,7,8,8a- Hexahydro-4H- pyrido[4,3- b][1,4]oxazin-3-one hydrochloride (BB 5A) and CAS RN 1260773-91-8 | 408.3 [M + H]⁺ |
| 12 | (−)- or (+)-trans-6-[3- (4-Phenoxyphenyl) azetidine- 1-carbonyl]- 4,4a,5,7,8,8a- hexahydropyrido[4,3- b][1,4]oxazin-3-one | 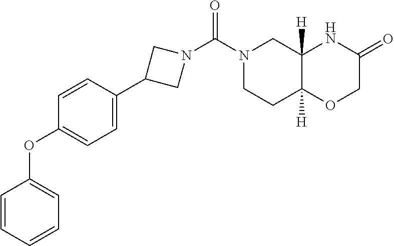 [SMILES: O=C1CO[C@H]2CCN(C(=O)N3CC(c4ccc(Oc5ccccc5)cc4)C3)C[C@@H]2N1] | (−)-trans- 4a,5,6,7,8,8a- Hexahydro-4H- pyrido[4,3- b][1,4]oxazin-3-one hydrochloride (BB 5B) and CAS RN 1260773-91-8 | 408.3 [M + H]⁺ |
| 13 | (+)- or (−)-trans-6-[3- [4-(2,4- Difluorophenyl) phenyl] azetidine-1- carbonyl]- 4,4a,5,7,8,8a- hexahydropyrido[4,3- b][1,4]oxazin-3-one | 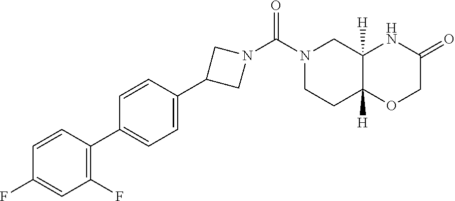 [SMILES: O=C1CO[C@@H]2CCN(C(=O)N3CC(c4ccc(-c5ccc(F)cc5F)cc4)C3)C[C@H]2N1] | (+)-trans- 4a,5,6,7,8,8a- Hexahydro-4H- pyrido[4,3- b][1,4]oxazin-3-one hydrochloride (BB 5A) and 3-[2-[2- Fluoro-4- (trifluoromethyl) phenyl] ethyl]azetidine 4- methylbenzenesulfonic acid (BB 7) | 428.4 [M + H]⁺ |
| 14 | (−)- or (+)-trans-6-[3- [4-(2,4- Difluorophenyl) phenyl] azetidine-1- carbonyl]- 4,4a,5,7,8,8a- hexahydropyrido[4,3- b][1,4]oxazin-3-one | 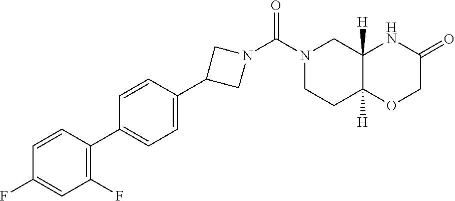 [SMILES: O=C1CO[C@H]2CCN(C(=O)N3CC(c4ccc(-c5ccc(F)cc5F)cc4)C3)C[C@@H]2N1] | (−)-trans- 4a,5,6,7,8,8a- Hexahydro-4H- pyrido[4,3- b][1,4]oxazin-3-one hydrochloride (BB 5B) and 3-[2-[2- Fluoro-4- (trifluoromethyl) phenyl] ethyl]azetidine 4- methylbenzenesulfonic acid (BB 7) | 428.3 [M + H]⁺ |
| 15 | (+)- or (−)-trans-6-[3- [4-(2,2,2- Trifluoroethyl) phenyl] azetidine-1- carbonyl]- 4,4a,5,7,8,8a- hexahydropyrido[4,3- b][1,4]oxazin-3-one | 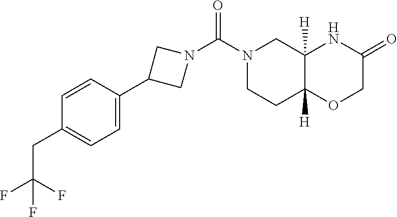 [SMILES: O=C1CO[C@@H]2CCN(C(=O)N3CC(c4ccc(CC(F)(F)F)cc4)C3)C[C@H]2N1] | (+)-trans- 4a,5,6,7,8,8a- Hexahydro-4H- pyrido[4,3- b][1,4]oxazin-3-one hydrochloride (BB 5A) and 3-[4-(2,2,2- Trifluoroethyl)phenyl] azetidine 4- methylbenzenesulfonic acid (BB 8) | 398.3 [M + H]⁺ |
| 16 | (−)- or (+)-trans-6-[3- [4-(2,2,2- Trifluoroethyl) phenyl] azetidine-1- carbonyl]- 4,4a,5,7,8,8a- hexahydropyrido[4,3- b][1,4]oxazin-3-one | 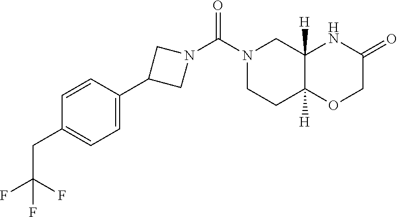 [SMILES: O=C1CO[C@H]2CCN(C(=O)N3CC(c4ccc(CC(F)(F)F)cc4)C3)C[C@@H]2N1] | (−)-trans- 4a,5,6,7,8,8a- Hexahydro-4H- pyrido[4,3- b][1,4]oxazin-3-one hydrochloride (BB 5B) and 3-[4-(2,2,2- Trifluoroethyl)phenyl] azetidine 4- methylbenzenesulfonic acid (BB 8) | 398.3 [M + H]⁺ |
| 17 | (+)- or (−)-trans-6-[6- [(2,4- Difluorophenyl) methyl]-2- azaspiro[3.3]heptane- 2-carbonyl]- 4,4a,5,7,8,8a- hexahydropyrido[4,3- | 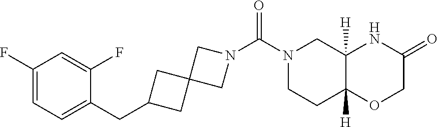 [SMILES: O=C1CO[C@@H]2CCN(C(=O)N3CC4(CC(Cc5ccc(F)cc5F)C4)C3)C[C@H]2N1] | (+)-trans- 4a,5,6,7,8,8a- Hexahydro-4H- pyrido[4,3- b][1,4]oxazin-3-one hydrochloride (BB 5A) and 6-[(2,4- Difluorophenyl) | 406.3 [M + H]⁺ |
| | b][1,4]oxazin-3-one | | methyl]-2- | |
| | | | azaspiro[3.3]heptane | |
| | | | 2,2,2-trifluoroacetic | |
| | | | acid (BB 9) | |
| 18 | (−)- or (+)-trans-6-[6- [(2,4- Difluorophenyl) methyl]-2- azaspiro[3.3]heptane- 2-carbonyl]- 4,4a,5,7,8,8a- hexahydropyrido[4,3- | 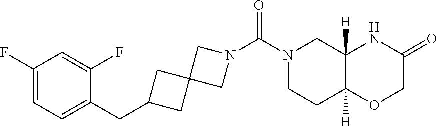 [SMILES: O=C1CO[C@H]2CCN(C(=O)N3CC4(CC(Cc5ccc(F)cc5F)C4)C3)C[C@@H]2N1] | (−)-trans- 4a,5,6,7,8,8a- Hexahydro-4H- pyrido[4,3- b][1,4]oxazin-3-one hydrochloride (BB 5B) and 6-[(2,4- Difluorophenyl) | 406.3 [M + H]⁺ |
| | b][1,4]oxazin-3-one | | methyl]-2- | |
| | | | azaspiro[3.3]heptane | |
| | | | 2,2,2-trifluoroacetic | |
| | | | acid (BB 9) | |
| 19 | (−)- or (+)-trans-6-[3- [6-(2- Chlorophenoxy)-3- pyridyl]azetidine-1- carbonyl]- 4,4a,5,7,8,8a- hexahydropyrido[4,3- b][1,4]oxazin-3-one | 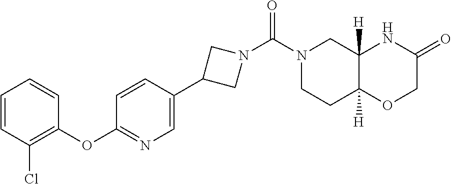 [SMILES: O=C1CO[C@H]2CCN(C(=O)N3CC(c4ccc(Oc5ccccc5Cl)nc4)C3)C[C@@H]2N1] | (−)-trans- 4a,5,6,7,8,8a- Hexahydro-4H- pyrido[4,3- b][1,4]oxazin-3-one hydrochloride (BB 5B) and 5-(Azetidin- 3-yl)-2-(2- chlorophenoxy) pyridine 4- methylbenzenesulfonic acid (BB 10) | 443.2 [M + H]⁺ |
| 20 | (+)- or (−)-trans-6-[3- [[2-Chloro-4- (trifluoromethyl) phenyl]methoxy] azetidine- 1-carbonyl]- 4,4a,5,7,8,8a- hexahydropyrido[4,3- b][1,4]oxazin-3-one | 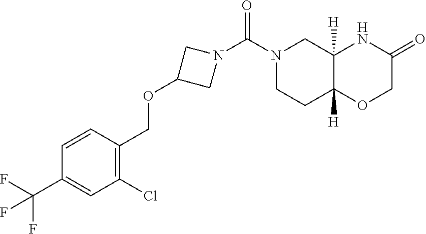 [SMILES: O=C1CO[C@@H]2CCN(C(=O)N3CC(OCc4ccc(C(F)(F)F)cc4Cl)C3)C[C@H]2N1] | (+)-trans- 4a,5,6,7,8,8a- Hexahydro-4H- pyrido[4,3- b][1,4]oxazin-3-one hydrochloride (BB 5A) and 3-[[2- Chloro-4- (trifluoromethyl) phenyl]methoxy] azetidine 2,2,2-trifluoroacetic acid (CAS RN 2411573-97-0) | 448.2 [M + H]⁺ |
| 21 | (−)- or (+)-trans-6-[3- [[2-Chloro-4- (trifluoromethyl) phenyl] methoxy]azetidine- 1-carbonyl]- 4,4a,5,7,8,8a- hexahydropyrido[4,3- b][1,4]oxazin-3-one | 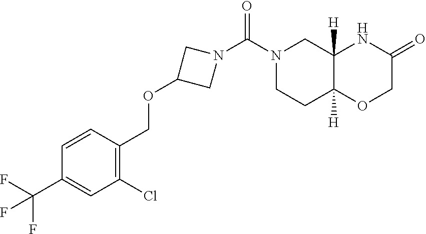 [SMILES: O=C1CO[C@H]2CCN(C(=O)N3CC(OCc4ccc(C(F)(F)F)cc4Cl)C3)C[C@@H]2N1] | (−)-trans- 4a,5,6,7,8,8a- Hexahydro-4H- pyrido[4,3- b][1,4]oxazin-3-one hydrochloride (BB 5B) and 3-[[2- Chloro-4- (trifluoromethyl) phenyl]methoxy] azetidine 2,2,2-trifluoroacetic acid (CAS RN 2411573-97-0) | 448.2 [M + H]⁺ |
| 22 | (+)- or (−)-trans-6-[3- [[4- (Trifluoromethoxy) phenyl]methoxy] azetidine-1-carbonyl]- 4,4a,5,7,8,8a- hexahydropyrido[4,3- b][1,4]oxazin-3-one | 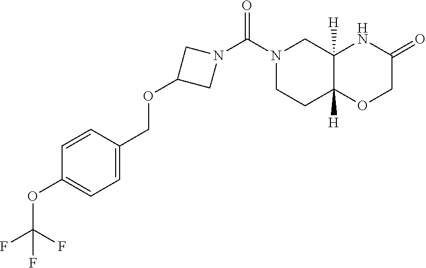 [SMILES: O=C1CO[C@@H]2CCN(C(=O)N3CC(OCc4ccc(OC(F)(F)F)cc4)C3)C[C@H]2N1] | (+)-trans- 4a,5,6,7,8,8a- Hexahydro-4H- pyrido[4,3- b][1,4]oxazin-3-one hydrochloride (BB 5A) and 3-[[4- (Trifluoromethoxy) phenyl]methoxy] azetidine (CAS RN 1121595-02-5) | 430.2 [M + H]⁺ |
| 23 | (−)- or (+)-trans-6-[3- [[4- (Trifluoromethoxy) phenyl]methoxy] azetidine-1-carbonyl]- 4,4a,5,7,8,8a- hexahydropyrido[4,3- b][1,4]oxazin-3-one | 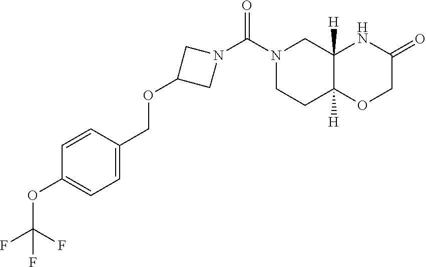 [SMILES: O=C1CO[C@H]2CCN(C(=O)N3CC(OCc4ccc(OC(F)(F)F)cc4)C3)C[C@@H]2N1] | (−)-trans- 4a,5,6,7,8,8a- Hexahydro-4H- pyrido[4,3- b][1,4]oxazin-3-one hydrochloride (BB 5B) and 3-[[4- (Trifluoromethoxy) phenyl]methoxy] azetidine (CAS RN 1121595-02-5) | 430.3 [M + H]⁺ |
| 24 | (+)- or (−)-trans-6-[4- [Bis(4- fluorophenyl)methyl] piperidine-1- carbonyl]- 4,4a,5,7,8,8a- hexahydropyrido[4,3- b][1,4]oxazin-3-one | 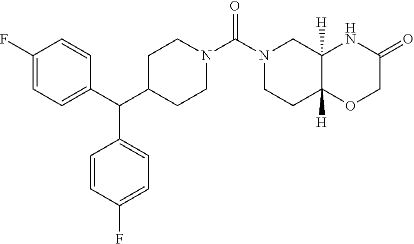 [SMILES: O=C1CO[C@@H]2CCN(C(=O)N3CCC(C(c4ccc(F)cc4)c4ccc(F)cc4)CC3)C[C@H]2N1] | (+)-trans- 4a,5,6,7,8,8a- Hexahydro-4H- pyrido[4,3- b][1,4]oxazin-3-one hydrochloride (BB 5A) and 4-(Bis(4- fluorophenyl) methyl)piperidine (CAS RN 60285-00-9) | 470.2 [M + H]⁺ |
| 25 | (−)- or (+)-trans-6-[4- [Bis(4- fluorophenyl)methyl] piperidine-1- carbonyl]- 4,4a,5,7,8,8a- hexahydropyrido[4,3- b][1,4]oxazin-3-one | 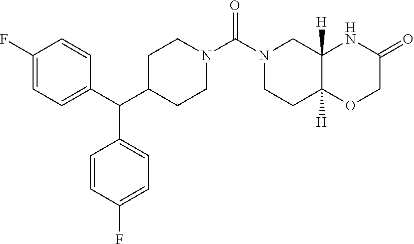 [SMILES: O=C1CO[C@H]2CCN(C(=O)N3CCC(C(c4ccc(F)cc4)c4ccc(F)cc4)CC3)C[C@@H]2N1] | (−)-trans- 4a,5,6,7,8,8a- Hexahydro-4H- pyrido[4,3- b][1,4]oxazin-3-one hydrochloride (BB 5B) and 4-(Bis(4- fluorophenyl) methyl)piperidine (CAS RN 60285-00-9) | 470.4 [M + H]⁺ |
| 26 | (+)- or (−)-trans-6-[2- [2-Fluoro-4- (trifluoromethyl) phenoxy]-7- azaspiro[3.5]nonane- 7-carbonyl]- 4,4a,5,7,8,8a- hexahydropyrido[4,3- b][1,4]oxazin-3-one | 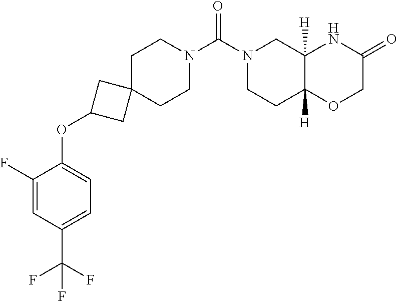 [SMILES: O=C1CO[C@@H]2CCN(C(=O)N3CCC4(CC3)CC(Oc3ccc(C(F)(F)F)cc3F)C4)C[C@H]2N1] | (+)-trans- 4a,5,6,7,8,8a- Hexahydro-4H- pyrido[4,3- b][1,4]oxazin-3-one hydrochloride (BB 5A) and 2-[2- Fluoro-4- (trifluoromethyl) phenoxy]-7- azaspiro[3.5]nonane 2,2,2-trifluoroacetic acid (BB 11) | 486.3 [M + H]⁺ |
| 27 | (−)- or (+)-trans-6-[2- [2-Fluoro-4- (trifluoromethyl) phenoxy]-7- azaspiro[3.5]nonane- 7-carbonyl]- 4,4a,5,7,8,8a- hexahydropyrido[4,3- b][1,4]oxazin-3-one | 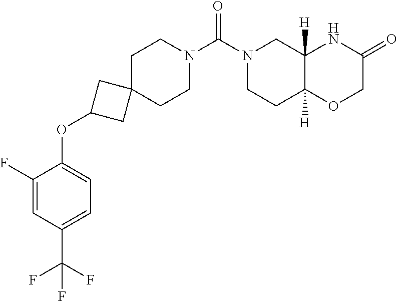 [SMILES: O=C1CO[C@H]2CCN(C(=O)N3CCC4(CC3)CC(Oc3ccc(C(F)(F)F)cc3F)C4)C[C@@H]2N1] | (−)-trans- 4a,5,6,7,8,8a- Hexahydro-4H- pyrido[4,3- b][1,4]oxazin-3-one hydrochloride (BB 5B) and 2-[2- Fluoro-4- (trifluoromethyl) phenoxy]-7- azaspiro[3.5]nonane 2,2,2-trifluoroacetic acid (BB 11) | 486.3 [M + H]⁺ |
| 28 | (+)- or (−)-trans-6-[4- [[4- (Trifluoromethyl) phenyl]methyl] piperidine- 1-carbonyl]- 4,4a,5,7,8,8a- hexahydropyrido[4,3- b][1,4]oxazin-3-one | 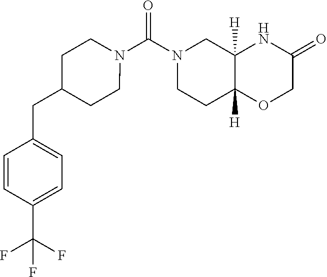 [SMILES: O=C1CO[C@@H]2CCN(C(=O)N3CCC(Cc4ccc(C(F)(F)F)cc4)CC3)C[C@H]2N1] | (+)-trans- 4a,5,6,7,8,8a- Hexahydro-4H- pyrido[4,3- b][1,4]oxazin-3-one hydrochloride (BB 5A) and 4-[[4- (Trifluoromethyl) phenyl]methyl] piperidine hydrochloride (CAS RN 193357-81-2) | 426.4 [M + H]⁺ |
| 29 | (−)- or (+)-trans-6-[4- [[4- (Trifluoromethyl) phenyl]methyl] piperidine- 1-carbonyl]- 4,4a,5,7,8,8a- hexahydropyrido[4,3- b][1,4]oxazin-3-one | 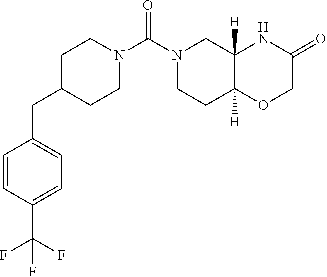 [SMILES: O=C1CO[C@H]2CCN(C(=O)N3CCC(Cc4ccc(C(F)(F)F)cc4)CC3)C[C@@H]2N1] | (−)-trans- 4a,5,6,7,8,8a- Hexahydro-4H- pyrido[4,3- b][1,4]oxazin-3-one hydrochloride (BB 5B) and 4-[[4- (Trifluoromethyl) phenyl]methyl] piperidine hydrochloride (CAS RN 193357-81-2) | 426.3 [M + H]⁺ |
| 30 | (−)- or (+)-trans-6-[4- [5-Chloro-1-(2- hydroxyethyl)indol- 3-yl]piperidine-1- carbonyl]- 4,4a,5,7,8,8a- hexahydropyrido[4,3- b][1,4]oxazin-3-one | 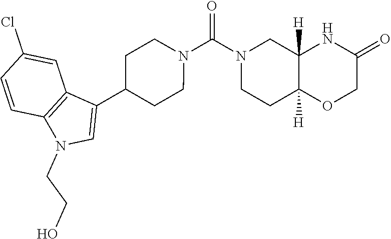 [SMILES: O=C1CO[C@H]2CCN(C(=O)N3CCC(c4cn(CCO)c5ccc(Cl)cc45)CC3)C[C@@H]2N1] | (−)-trans- 4a,5,6,7,8,8a- Hexahydro-4H- pyrido[4,3- b][1,4]oxazin-3-one hydrochloride (BB 5B) and 2-[5-Chloro- 3-(4-piperidyl)indol- 1-yl]ethanol (CAS RN 2377009-11-3) | 461.2 [M + H]⁺ |
| 31 | [3-[[2-Fluoro-4- (trifluoromethyl) phenyl]methoxy] azetidin-1-yl]- [rac-(4aR,8aR)- 2,3,4,4a,5,7,8,8a- octahydropyrido[4,3- b][1,4]oxazin-6- yl]methanone | 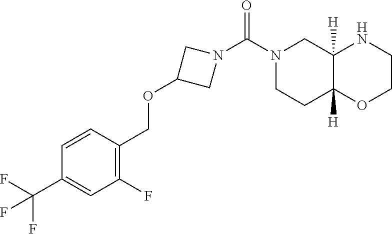 [SMILES: O=C(N1CC(OCc2ccc(C(F)(F)F)cc2F)C1)N1CC[C@H]2OCCN[C@@H]2C1] | rac-(4aR,8aR)- Octahydro-2H- pyrido[4,3- b][1,4]oxazine (CAS RN 1909294-04-7) and 3-[[2-Fluoro-4- (trifluoromethyl) phenyl]methoxy] azetidine 4- methylbenzenesulfonic acid (BB 4) | 418.3 [M + H]⁺ |

### Synthesis of Building Blocks

### BB 1

### rac-trans-Hexahydro-2H-pyrido[4,3-b][1,4]oxazin-3(4H)-one hydrochloride

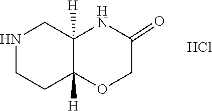 [SMILES: O=C1CO[C@@H]2CCNC[C@H]2N1 ; Cl]

### Step 1: tert-Butyl rac-trans-3-[(2-chloroacetyl)amino]-4-hydroxy-piperidine-1-carboxylate

To a suspension of trans-3-amino-1-boc-4-hydroxypiperidine (1.01 g, 4.69 mmol, 1.0 equiv; CAS RN 1268511-99-4) and sodium acetate trihydrate (1.28 g, 9.38 mmol, 2.0 equiv) in a mixture of acetone (8 mL) and water (1 mL) was added 2-chloroacetyl chloride (0.53 g, 0.37 mL, 4.69 mmol, 1.0 equiv) via syringe pump dropwise at rt over 3 h. The reaction mixture was evaporated and the crude product purified by silica gel chromatography using an MPLC system eluting with a gradient of n-heptane:EtOH/ethyl acetate (1:3) (70:30 to 10:90) to furnish the title compound as a colorless foam (0.44 g, 64%). MS (ESI): m/z=237.1 [M+2H-tBu]⁺.

### Step 2: tert-Butyl rac-trans-3-oxo-4,4a,5,7,8,8a-hexahydropyrido[4,3-b][1,4]oxazine-6-carboxylate

To an ice-cold solution of tert-butyl rac-trans-3-[(2-chloroacetyl)amino]-4-hydroxy-piperidine-1-carboxylate (1.18 g, 4.03 mmol, 1.0 equiv) in DCM (18 mL) was added dropwise a solution of potassium tert-butoxide (1.81 g, 16.1 mmol, 4.0 equiv) in 2-propanol (46 mL). The ice-bath was removed and the mixture was stirred at rt for 24 h while getting a white suspension. The reaction mixture was evaporated and the residue taken up in ethyl acetate and water. The aqueous layer was extracted twice with ethyl acetate. The combined organic layers were dried over MgSO₄, filtered and evaporated. The crude product was purified by silica gel chromatography using an MPLC system eluting with a gradient of DCM:methanol (100:0 to 90:10) to yield the title compound as a colorless foam (0.84 g, 75%). MS (ESI): m/z=201.1 [M+2H-tBu]⁺.

### Step 3: rac-trans-Hexahydro-2H-pyrido[4,3-b][1,4]oxazin-3(4H)-one hydrochloride

To a 2 M solution of HCl in diethylether (15.5 mL, 31.0 mmol, 10 equiv) was added tert-butyl rac-trans-3-oxo-4,4a,5,7,8,8a-hexahydropyrido[4,3-b][1,4]oxazine-6-carboxylate (0.80 g, 3.11 mmol, 1.0 equiv) and the reaction mixture stirred at rt for 24 h. The colorless suspension was cooled down in the fridge to 0° C. for 2 h, the precipitate filtered, washed with diethylether and dried under vacuum. The title compound was obtained as a colorless solid (0.62 g, 98%). MS (ESI): m/z=157.1 [M+H]⁺.

### BB 2

### 3-(4-(tert-Butyl)phenyl)azetidine 4-methylbenzenesulfonate

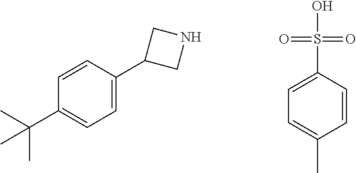

To a solution of tert-butyl 3-(4-tert-butylphenyl)azetidine-1-carboxylate (1.8 g, 6.22 mmol, 1.0 equiv; CAS RN 1629889-13-9) in ethyl acetate (15 mL) was added 4-methylbenzenesulfonic acid hydrate (1.66 g, 8.70 mmol, 1.4 equiv) and the mixture was heated at reflux for 12 h. The solution was evaporated to get the title compound as a brown oil (1.69 g, 66%). MS (ESI): m/z=190.2 [M+H−Ts]⁺.

### BB 3

### 3-[4-[1-(Trifluoromethyl)cyclopropyl]phenyl]azetidine 4-methylbenzenesulfonate

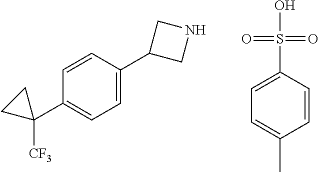 [SMILES: FC(F)(F)C1(c2ccc(C3CNC3)cc2)CC1 ; Cc1ccc(S(=O)(=O)O)cc1]

### Step 1: tert-Butyl 3-[4-[1-(trifluoromethyl)cyclopropyl]phenyl]azetidine-1-carboxylate

To a 20 mL vial, equipped with a stir bar, was added 1-bromo-4-(1-(trifluoromethyl)cyclopropyl)benzene (561 mg, 2.12 mmol, 1.0 equiv; CAS RN 1227160-18-0), tert-butyl 3-iodoazetidine-1-carboxylate (600 mg, 2.12 mmol, 1.0 equiv; CAS RN 254454-54-1), tris(trimethylsilyl)silane (527 mg, 653 μL, 2.12 mmol, 1.0 equiv), photocatalyst bis[3,5-difluoro-2-[5-(trifluoromethyl)-2-pyridyl]phenyl]iridium(1+) 4-tert-butyl-2-(4-tert-butyl-2-pyridyl)pyridine hexafluorophosphate (23.8 mg, 21.2 μmol, 0.01 equiv; Ir[dF(CF3)ppy]2(dtbbpy))PF6, CAS RN 870987-63-6) and anhydrous sodium carbonate (449 mg, 4.24 mmol, 2.0 equiv). The vial was sealed and placed under Ar before dimethoxyethane (9 mL) was added. To a separate vial was added nickel(II) chloride ethylene glycol dimethyl ether complex (4.65 mg, 21.2 μmol, 0.01 equiv; CAS RN 29046-78-4) and 4,4′-di-tert-butyl-2,2′-bipyridine (5.68 mg, 21.2 μmol, 0.01 equiv). The vial was sealed, purged with Ar, and dimethoxyethane (4 mL) was added. The precatalyst solution was sonicated for 5 min, after which 2 mL were syringed into the reaction vessel. The reaction mixture was degassed with Ar and irradiated with a blue LED lamp (420 nm) for 1 h. The reaction was quenched by exposure to air, filtered and the solvent evaporated. The crude reaction mixture was purified by silica gel chromatography using an MPLC system eluting with a gradient of n-heptane:ethyl acetate (100:0 to 70:30) to furnish the title compound as a colorless solid (0.51 g, 66%). MS (ESI): m/z=286.1 [M+2H-tBu]⁺.

### Step 2: 3-[4-[1-(Trifluoromethyl)cyclopropyl]phenyl]azetidine 4-methylbenzenesulfonate

To a solution of tert-butyl 3-[4-[1-(trifluoromethyl)cyclopropyl]phenyl]azetidine-1-carboxylate (0.5 g, 1.46 mmol, 1.0 equiv) in ethyl acetate (5 mL) was added 4-methylbenzenesulfonic acid hydrate (0.29 g, 1.54 mmol, 1.1 equiv) and the mixture was heated at reflux for 2 h. The suspension was cooled in the fridge at 0° C. for 1 h and the filtered. The precipitate was washed with ethyl acetate and dried to yield the title compound as a colorless solid (0.52 g, 82%). MS (ESI): m/z=242.2 [M+H]⁺.

### BB 4

### 3-[[2-Fluoro-4-(trifluoromethyl)phenyl]methoxy]azetidine 4-methylbenzenesulfonic Acid

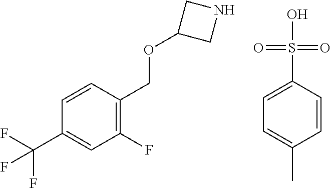 [SMILES: Fc1cc(C(F)(F)F)ccc1COC1CNC1 ; Cc1ccc(S(=O)(=O)O)cc1]

### Step 1: tert-Butyl 3-[[2-fluoro-4-(trifluoromethyl)phenyl]methoxy]azetidine-1-carboxylate

To an ice-cold solution of tert-butyl 3-hydroxyazetidine-1-carboxylate (2.02 g, 11.7 mmol, 1.0 equiv) in DMF (25 mL) was added sodium hydride (0.56 g, 12.8 mmol, 1.1 equiv; 55% in mineral oil) in portions and the reaction mixture was stirred for 30 min. A solution of 1-(bromomethyl)-2-fluoro-4-(trifluoromethyl)benzene (3.0 g, 11.7 mmol, 1.0 equiv) in DMF (5 mL) was added dropwise to the reaction mixture and stirring continued at rt for 3 h. The reaction mixture was poured on a mixture of a sat. aqueous NH₄Cl solution (70 mL) and ethyl acetate (70 mL) and the aqueous layer was extracted twice with ethyl acetate. The combined organic layers were dried over MgSO₄, filtered and evaporated. The crude product was purified by silica gel chromatography using an MPLC system eluting with a gradient of n-heptane:ethyl acetate (100:0 to 60:40) to yield the title compound as a light yellow oil (3.66 g, 90%). MS (ESI): m/z=294.1 [M+2H-tBu]⁺.

### Step 2: 3-[[2-Fluoro-4-(trifluoromethyl)phenyl]methoxy]azetidine 4-methylbenzenesulfonic Acid

To a solution of tert-butyl 3-[[2-fluoro-4-(trifluoromethyl)phenyl]methoxy]azetidine-1-carboxylate (7.8 g, 22.3 mmol, 1.0 equiv) in ethyl acetate (130 mL) was added 4-methylbenzenesulfonic acid hydrate (4.61 g, 26.8 mmol, 1.2 equiv) and the mixture was heated at reflux for 2 h. The suspension was cooled in the fridge at 0° C. for 1 h and filtered. The precipitate was washed with ethyl acetate and dried to yield the title compound as a colorless solid (7.3 g, 81%). MS (ESI): m/z=250.2 [M+H]⁺.

### BB 5A and BB 5B

### (+)-trans-4a,5,6,7,8,8a-Hexahydro-4H-pyrido[4,3-b][1,4]oxazin-3-one hydrochloride and (−)-trans-4a,5,6,7,8,8a-Hexahydro-4H-pyrido[4,3-b][1,4]oxazin-3-one hydrochloride

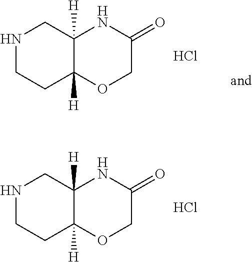 [SMILES: O=C1CO[C@@H]2CCNC[C@H]2N1 ; Cl ; O=C1CO[C@H]2CCNC[C@@H]2N1 ; Cl]

### Step 1: (+)-tert-Butyl trans-3-oxo-4,4a,5,7,8,8a-hexahydropyrido[4,3-b][1,4]oxazine-6-carboxylate and (−)-tert-Butyl trans-3-oxo-4,4a,5,7,8,8a-hexahydropyrido[4,3-b][1,4]oxazine-6-carboxylate

The enantiomers of tert-butyl rac-trans-3-oxo-4,4a,5,7,8,8a-hexahydropyrido[4,3-b][1,4]oxazine-6-carboxylate (3.93 g, 13.4 mmol; BB 1, step 2) were separated by SFC (preparative: Chiralpak AD-H column, 220 nm, 5 μm, 250×20 mm; analytical: Chiralpak AD-H column, 220 nm, 5 μm, 150×4.6 mm) using MeOH (20-40%) as a cosolvent.

Second eluting enantiomer: (−)-tert-butyl trans-3-oxo-4,4a,5,7,8,8a-hexahydropyrido[4,3-b][1,4]oxazine-6-carboxylate. Off-white foam (1.0 g, 81%). Analytical SFC: t_{R}=2.49 min. [α]^{D}₂₀=−16.3° (c=1.0 in MeOH). MS (ESI): m/z=201.1 [M+2H-tBu]⁺.

First eluting enantiomer: (+)-tert-butyl trans-3-oxo-4,4a,5,7,8,8a-hexahydropyrido[4,3-b][1,4]oxazine-6-carboxylate. Off-white foam (1.2 g, 92%). Analytical SFC: t_{R}=1.36 min. [α]^{D}₂₀=+19.10 (c=1.0 in MeOH). MS (ESI): m/z=201.1 [M+2H-tBu]⁺.

### Step 2: (+)-trans-4a,5,6,7,8,8a-Hexahydro-4H-pyrido[4,3-b][1,4]oxazin-3-one hydrochloride (BB 5A) and (−)-trans-4a,5,6,7,8,8a-Hexahydro-4H-pyrido[4,3-b][1,4]oxazin-3-one hydrochloride (BB 5B)

To a solution of (−)-tert-butyl trans-3-oxo-4,4a,5,7,8,8a-hexahydropyrido[4,3-b][1,4]oxazine-6-carboxylate (1.0 g, 3.89 mmol, 1.0 equiv) in DCM (10 mL) was added a 4 M solution of HCl in dioxan (9.7 mL, 38.9 mmol, 10 equiv) and the reaction mixture stirred at 5° C. for 1 h and then warmed up to rt. After 16 h, the solvent is evaporated, the white precipitate filtered, washed with diethylether and dried under vacuum. The title compound was obtained as a colorless solid (0.74 g, 99%). [α]^{D}₂₀=+32.9° (c=1.0 in MeOH). MS (ESI): m/z=157.1 [M+H]⁺.

To a solution of (+)-tert-butyl trans-3-oxo-4,4a,5,7,8,8a-hexahydropyrido[4,3-b][1,4]oxazine-6-carboxylate (1.1 g, 4.31 mmol, 1.0 equiv) in DCM (10 mL) was added a 4 M solution of HCl in dioxan (10.8 mL, 43.1 mmol, 10 equiv) and the reaction mixture stirred at 5° C. for 1 h and then warmed up to rt. After 16 h, the solvent is evaporated, the white precipitate filtered, washed with diethylether and dried under vacuum. The title compound was obtained as a colorless solid (0.82 g, 99%). [α]^{D}₂₀=−31.8° (c=1.0 in MeOH). MS (ESI): m/z=157.1 [M+H]⁺.

### BB 6

### 3-[2-[2-Fluoro-4-(trifluoromethyl)phenyl]ethyl]azetidine 4-methylbenzenesulfonic acid

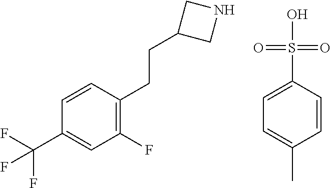 [SMILES: Fc1cc(C(F)(F)F)ccc1CCC1CNC1 ; Cc1ccc(S(=O)(=O)O)cc1]

### Step 1: Diethyl (2-fluoro-4-(trifluoromethyl)benzyl)phosphonate

A solution of 1-(bromomethyl)-2-fluoro-4-(trifluoromethyl)benzene (1.1 g, 4.28 mmol, 1.0 equiv; CAS RN 239087-07-1) in triethyl phosphite (1.78 g, 1.83 mL, 10.7 mmol; 2.5 equiv) was stirred at reflux for 3 h. The crude reaction mixture was purified by silica gel chromatography using an MPLC system eluting with a gradient of n-heptane:ethyl acetate (100:0 to 0:100) to furnish the title compound as a colorless oil (0.83 g, 62%). MS (ESI): m/z=315.2 [M+H]⁺.

### Step 2: tert-Butyl 3-[(E)-2-[2-fluoro-4-(trifluoromethyl)phenyl]vinyl]azetidine-1-carboxylate

To an ice-cold suspension of sodium hydride (122 mg, 2.8 mmol, 1.1 equiv; 55% in mineral oil) in THF (5 mL) was added diethyl (2-fluoro-4-(trifluoromethyl)benzyl)phosphonate (800 mg, 2.55 mmol, 1.0 equiv) in THF (5 mL) within 5 min and the mixture was stirred at this temperature for 30 min. To the light brown mixture was added dropwise a solution of tert-butyl 3-formylazetidine-1-carboxylate (472 mg, 2.55 mmol, 1.0 equiv) in THF (2.5 mL) and stirring of the reaction mixture continued at 0-6° C. for 3 h. The reaction mixture was poured into water and ethyl acetate and the layers were separated. The organic layer was washed once with brine, dried over MgSO4, filtered, treated with silica gel and evaporated. The compound was purified by silica gel chromatography using an MPLC system eluting with a gradient of n-heptane:ethyl acetate (100:0 to 50:50) to get the title compound as a colorless oil (0.61 g, 69%). MS (ESI): m/z=290.1 [M+2H-tBu]⁺.

### Step 3: tert-Butyl 3-[2-[2-fluoro-4-(trifluoromethyl)phenyl]ethyl]azetidine-1-carboxylate

To a solution of tert-butyl 3-[(E)-2-[2-fluoro-4-(trifluoromethyl)phenyl]vinyl]azetidine-1-carboxylate (607 mg, 1.76 mmol, 1.0 equiv) in a mixture of MeOH (7 mL) and ethyl acetate (7 mL) was added Pd/C 10% (60 mg, 1.76 mmol, 1.0 equiv) and the reaction mixture was stirred under an atmosphere of hydrogen (1 bar) at rt for 4 h. The suspension was filtered through a Celite pad, washed with ethyl acetate and dried under vacuum. The title compound was obtained as a colorless oil (0.61 g, 98%). MS (ESI): m/z=292.1 [M+2H-tBu]⁺.

### Step 4: 3-[2-[2-Fluoro-4-(trifluoromethyl)phenyl]ethyl]azetidine 4-methylbenzenesulfonic Acid

To a solution of tert-butyl 3-[2-[2-fluoro-4-(trifluoromethyl)phenyl]ethyl]azetidine-1-carboxylate (111 mg, 0.32 mmol, 1.0 equiv) in ethyl acetate (1.2 mL) was added 4-methylbenzenesulfonic acid hydrate (66 mg, 0.38 mmol, 1.2 equiv) and the mixture was heated at reflux for 2 h. The suspension was cooled in the fridge at 0° C. for 1 h and filtered. The precipitate was washed with ethyl acetate and dried to yield the title compound as a colorless solid (96 mg, 72%). MS (ESI): m/z=248.2 [M+H]⁺.

### BB 7

### 3-[2-[2-Fluoro-4-(trifluoromethyl)phenyl]ethyl]azetidine 4-methylbenzenesulfonic Acid

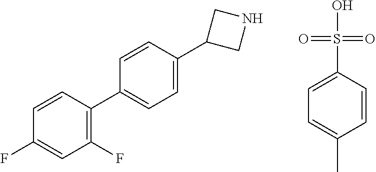 [SMILES: Fc1ccc(-c2ccc(C3CNC3)cc2)c(F)c1 ; Cc1ccc(S(=O)(=O)O)cc1]

### Step 1: tert-Butyl 3-(4-bromophenyl)azetidine-1-carboxylate

To a suspension of tert-butyl 3-iodoazetidine-1-carboxylate (2.0 g, 7.06 mmol, 1.0 equiv; CAS RN 254454-54-1) and (4-bromophenyl)boronic acid (2.84 g, 14.1 mmol, 2.0 equiv; CAS RN 5467-74-3) in 2-propanol (25 mL) was added rac-trans-2-aminocyclohexan-1-ol (48.8 mg, 424 μmol, 0.06 equiv), nickel(II) iodide (132 mg, 424 μmol, 0.06 equiv) and sodium bis(trimethylsilyl)amide (6.48 g, 14.1 mmol, 2.0 equiv; 40% in THF) at rt under Ar. The reaction mixture was heated by microwave irradiation to 80° C. for 30 min. The mixture was then poured on water and ethyl acetate (contains an insoluble solid) and the aqueous layer extracted twice with ethyl acetate. The organic layers were dried over MgSO₄, filtered, treated with silica gel and evaporated. The compound was purified by silica gel chromatography using an MPLC system eluting with a gradient of n-heptane:ethyl acetate (100:0 to 50:50) to provide the title compound as a colorless oil (1.33 g, 60%). MS (ESI): m/z=256.0 [M+2H-tBu]⁺.

### Step 2: tert-Butyl 3-[4-(2,4-difluorophenyl)phenyl]azetidine-1-carboxylate

A suspension of tert-butyl 3-(4-bromophenyl)azetidine-1-carboxylate (1.3 g, 4.16 mmol, 1.0 equiv), (2,4-difluorophenyl)boronic acid (658 mg, 4.16 mmol, 1.0 equiv; CAS RN 144025-03-6), potassium carbonate (2.88 g, 20.8 mmol, 5.0 equiv), tetrakis(triphenylphosphine)palladium (0) (241 mg, 208 μmol, 0.05 equiv) in a mixture of THF (10 mL) and water (1 mL) was heated by microwave irradiation to 110° C. for 15 min. The mixture was then poured on water and ethyl acetate and the aqueous layer extracted three times with ethyl acetate. The organic layers were dried over MgSO₄, filtered, treated with silica gel and evaporated. The compound was purified by silica gel chromatography using an MPLC system eluting with a gradient of n-heptane:ethyl acetate (100:0 to 50:50) to yield the title compound as a yellow oil (1.20 g, 79%). MS (ESI): m/z=290.2 [M+2H-tBu]⁺.

### Step 3: 3-[2-[2-Fluoro-4-(trifluoromethyl)phenyl]ethyl]azetidine 4-methylbenzenesulfonic Acid

To a solution of tert-butyl 3-[4-(2,4-difluorophenyl)phenyl]azetidine-1-carboxylate (1.20 g, 3.47 mmol, 1.0 equiv) in ethyl acetate (5 mL) was added 4-methylbenzenesulfonic acid hydrate (0.72 g, 4.17 mmol, 1.2 equiv) and the mixture was heated at reflux for 2 h. The suspension was cooled in the fridge at 0° C. for 1 h and filtered. The precipitate was washed with ethyl acetate and dried to yield the title compound as a colorless solid (0.92 g, 63%). MS (ESI): m/z=246.2 [M+H]⁺.

### BB 8

### 3-[4-(2,2,2-Trifluoroethyl)phenyl]azetidine 4-methylbenzenesulfonic Acid

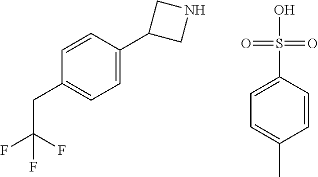

### Step 1: tert-Butyl 3-[4-(2,2,2-trifluoroethyl)phenyl]azetidine-1-carboxylate

The product was obtained in analogy to BB3/Step 1 from 1-bromo-4-(2,2,2-trifluoroethyl)benzene (CAS RN 155820-88-5) as a colorless oil. MS (ESI): m/z=260.1 [M+2H-tBu]⁺.

### Step 2: 3-[4-(2,2,2-Trifluoroethyl)phenyl]azetidine 4-methylbenzenesulfonic Acid

To a solution of tert-butyl 3-[4-(2,2,2-trifluoroethyl)phenyl]azetidine-1-carboxylate (0.98 g, 3.09 mmol, 1.0 equiv) in ethyl acetate (12 mL) was added 4-methylbenzenesulfonic acid hydrate (0.64 g, 3.71 mmol, 1.2 equiv) and the mixture was heated at reflux for 2 h. The suspension was cooled in the fridge at 0° C. for 1 h and filtered. The precipitate was washed with ethyl acetate and dried to yield the title compound as a colorless solid (0.54 g, 45%). MS (ESI): m/z=216.1 [M+H]⁺.

### BB 9

### 6-[(2,4-Difluorophenyl)methyl]-2-azaspiro[3.3]heptane 2,2,2-trifluoroacetic Acid

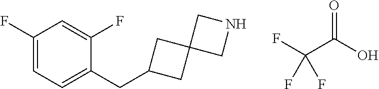 [SMILES: Fc1ccc(CC2CC3(CNC3)C2)c(F)c1 ; O=C(O)C(F)(F)F]

### Step 1: (2,4-Difluorobenzyl)triphenylphosphonium Bromide

To a solution of triphenylphosphine (1.27 g, 4.83 mmol, 1.0 equiv) in ACN (10 mL) was added 1-(bromomethyl)-2,4-difluorobenzene (1.0 g, 4.83 mmol, 1.0 equiv; CAS RN 23915-07-3) under Ar. The reaction mixture was stirred at 80° C. for 3 h and then allowed to cool to rt. tert-Butyl methyl ether (100 mL) was added and the suspension stirred at rt for 30 min. The solid was filtered off, washed with tert-butyl methyl ether and the solid dried. The title compound was obtained as a white solid (2.02 g, 98%). MS (ESI): m/z=439.2 [M+H]⁺.

### Step 2: tert-Butyl 6-[(2,4-difluorophenyl)methylene]-2-azaspiro[3.3]heptane-2-carboxylate

To a solution of (2,4-difluorobenzyl)triphenylphosphonium bromide (1.7 g, 3.62 mmol, 1.0 equiv) in dry THF (10 mL) was added LiHMDS (7.24 mL, 7.24 mmol, 2.0 equiv; 1 M solution in THF) at −78° C. under Ar and the reaction mixture stirred for 2 h. Then at rt, tert-butyl 6-oxo-2-azaspiro[3.3]heptane-2-carboxylate (1.53 g, 7.24 mmol, 2.0 equiv; CAS RN 1181816-12-5) was added and the mixture stirred at 85° C. overnight. tert-Butyl methyl ether was added and the precipitate (triphenylphosphine oxide) filtered off. The filtrate was concentrated and purified by silica gel chromatography using an MPLC system eluting with a gradient of n-heptane:ethyl acetate (100:0 to 70:30) to yield the title compound as a white solid (0.35 g, 30%). MS (ESI): m/z=266.2 [M+2H-tBu]⁺.

### Step 3: tert-Butyl 6-[(2,4-difluorophenyl)methyl]-2-azaspiro[3.3]heptane-2-carboxylate

To a solution of tert-butyl 6-[(2,4-difluorophenyl)methylene]-2-azaspiro[3.3]heptane-2-carboxylate (0.35 g, 1.09 mmol, 1.0 equiv) in ethyl acetate (10 mL) was added Pd/C 10% (116 mg, 0.11 mmol, 0.1 equiv) and the reaction mixture was stirred under an atmosphere of hydrogen (1 bar) at rt for 2 h. The suspension was filtered through a Celite pad, washed with ethyl acetate and dried under vacuum. The title compound was obtained as a white solid (0.35 g, 98%). MS (ESI): m/z=268.2 [M+2H-tBu]⁺.

### Step 4: 6-[(2A-Difluorophenyl)methyl]-2-azaspiro[3.3]heptane 2,2,2-trifluoroacetic Acid

To a solution of tert-butyl 6-[(2,4-difluorophenyl)methyl]-2-azaspiro[3.3]heptane-2-carboxylate (55 mg, 170 μmol, 1.0 equiv) in DCM (3 mL) was added TFA (78 mg, 52 μl, 680 μmol, 4.0 equiv). The resultant reaction mixture was stirred at rt for 2 h and was then concentrated in vacuo (azeotrop with toluene). The title compound was obtained as a colorless oil and used in the next step without further purification (58 mg, quant). MS (ESI): m/z=224.2 [M+H]⁺.

### BB 10

### 5-(Azetidin-3-yl)-2-(2-chlorophenoxy)pyridine 4-methylbenzenesulfonic Acid

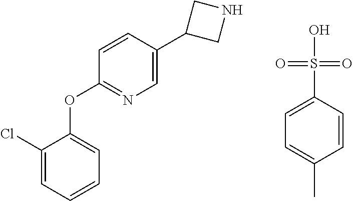 [SMILES: Clc1ccccc1Oc1ccc(C2CNC2)cn1 ; Cc1ccc(S(=O)(=O)O)cc1]

### Step 1: tert-Butyl 3-(6-(2-chlorophenoxy)pyridin-3-yl)azetidine-1-carboxylate

The product was obtained in analogy to BB 3/Step 1 starting from 5-bromo-2-(2-chlorophenoxy)pyridine (CAS RN 1240670-82-9) and tert-butyl 3-bromoazetidine-1-carboxylate (CAS RN 1064194-10-0) to get the desired compound as a yellow oil (0.44 g, 48%). MS (ESI): m/z=361.2 [M+H]⁺.

### Step 2: 5-(Azetidin-3-yl)-2-(2-chlorophenoxy)pyridine 4-methylbenzenesulfonic Acid

To a solution of tert-butyl 3-(6-(2-chlorophenoxy)pyridin-3-yl)azetidine-1-carboxylate (436 mg, 1.21 mmol, 1.0 equiv) in ethyl acetate (6 mL) was added 4-methylbenzenesulfonic acid hydrate (237 mg, 1.24 mmol, 1.03 equiv) and the mixture was heated at reflux for 18 h. The suspension was cooled in the fridge at 0° C. for 1 h and filtered. The precipitate was washed with diethylether and dried to yield the title compound as a white solid (470 mg, 89%). MS (ESI): m/z=261.1 [M+H]⁺.

### BB 11

### 2-[2-Fluoro-4-(trifluoromethyl)phenoxy]-7-azaspiro[3.5]nonane 2,2,2-trifluoroacetic Acid

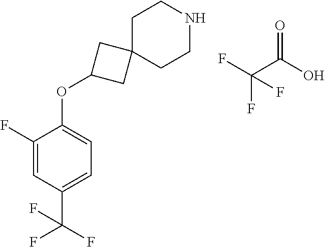

### Step 1: tert-Butyl 2-[2-fluoro-4-(trifluoromethyl)phenoxy]-7-azaspiro[3.5]nonane-7-carboxylate

To a solution of tert-butyl 2-hydroxy-7-azaspiro[3.5]nonane-7-carboxylate (442 mg, 1.83 mmol, 1.0 equiv; CAS RN 240401-28-9) in THF (8 mL) was added 2-fluoro-4-(trifluoromethyl)phenol (330 mg, 1.83 mmol, 1.0 equiv; CAS RN 77227-78-2) and triphenylphosphine (529 mg, 2.02 mmol, 1.1 equiv). After stirring at rt for 5 min, the solution was cooled down in an ice-bath and DEAD (351 mg, 319 μl, 2.02 mmol, 1.1 equiv) was added dropwise over 10 min. After stirring for 1 h in an ice-bath, stirring of the mixture was continued at rt for 5 h. The reaction mixture was poured on water and diethylether and the layers were separated. The organic layer was washed with water, aqueous NaOH (1 M) solution and brine, dried over MgSO₄, filtered and evaporated. The crude product was purified by silica gel chromatography using an MPLC system eluting with a gradient of n-heptane:ethyl acetate (100:0 to 60:40) to get the title compound as a colorless solid (0.63 g, 85%). MS (ESI): m/z=348.1 [M+2H-tBu]⁺.

### Step 2: 2-[2-Fluoro-4-(trifluoromethyl)phenoxy]-7-azaspiro[3.5]nonane 2,2,2-trifluoroacetic Acid

To a solution of tert-butyl 2-[2-fluoro-4-(trifluoromethyl)phenoxy]-7-azaspiro[3.5]nonane-7-carboxylate (70 mg, 174 μmol, 1.0 equiv) in DCM (1 mL) was added TFA (66.8 μl, 868 μmol, 5.0 equiv) and the mixture was stirred at rt for 20 h. The solution was evaporated to get the title compound as colorless solid (73 mg, 100%). MS (ESI): m/z=304.2 [M+H]⁺.

### Example 32

A compound of formula (Ia) or (Ib) can be used in a manner known per se as the active ingredient for the production of tablets of the following composition:

### Per Tablet

| | | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example 33

A compound of formula (Ia) or (Ib) can be used in a manner known per se as the active ingredient for the production of capsules of the following composition:

### Per Capsule

| | | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula (Ia) or (Ib) 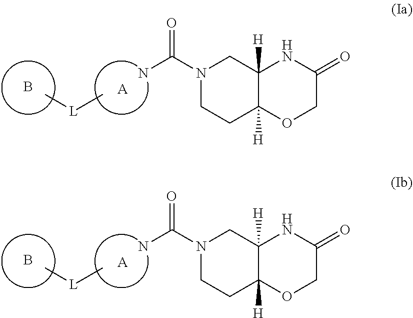
or a pharmaceutically acceptable salt thereof,
wherein:
A is a 3-14 membered heterocycle substituted with R^{A};
B is C₆-C₁₄-aryl or 5-14 membered heteroaryl substituted with R¹, R² and R³;
L is selected from a covalent bond, —C≡C—, —CHR^{L}—, —CH₂CHR^{L}—, —O—, —OCH₂—, and —CH₂O—; and
R¹, R², and R³ are independently selected from hydrogen, halogen, cyano, C₁-C₆-alkylsulfonyl, R^{b}R^{c}N, C₁-C₆-alkyl, C₁-C₆-alkoxy, halo-C₁-C₆-alkyl, halo-C₁-C₆-alkoxy, hydroxy-C₁-C₆-alkyl, C₆-C₁₄-aryl, C₃-C₁₀-cycloalkyl, 3-14 membered heterocyclyl, 5-14 membered heteroaryl, C₆-C₁₄-aryloxy, C₃-C₁₀-cycloalkyloxy, 3-14 membered heterocyclyloxy, and 5-14 membered heteroaryloxy, wherein said C₃-C₁₀-cycloalkyl, C₆-C₁₄-aryl, 3-14 membered heterocyclyl, 5-14 membered heteroaryl, C₆-C₁₄-aryloxy, C₃-C₁₀-cycloalkyloxy, 3-14 membered heterocyclyloxy, and 5-14 membered heteroaryloxy are optionally substituted with one or more substituents that are independently selected from halogen, C₁-C₆-alkyl, halo-C₁-C₆-alkyl, C₁-C₆-alkoxy, halo-C₁-C₆-alkoxy, and carbamoyl;
R^{A} is selected from hydrogen and C₁-C₆-alkyl;
R^{b} and R^{c} are independently selected from hydrogen, C₁-C₆-alkyl and C₆-C₁₄-aryl; and
R^{L} is selected from hydrogen, C₁-C₆-alkyl, hydroxy-C₁-C₆-alkyl, alkoxy-C₁-C₆-alkyl, halo-C₁-C₆-alkyl, C₆-C₁₄-aryl, and halo-C₆-C₁₄-aryl.

2. The compound of formula (Ia) or (Ib) according to claim 1, or a pharmaceutically acceptable salt thereof, wherein the compound of formula (Ia) or (Ib) is not selected from:
(4aS,8aS)-6-[4-[[4-(trifluoromethyl)phenyl]methyl]piperidine-1-carbonyl]-4,4a,5,7,8,8a-hexahydropyrido[4,3-b][1,4]oxazin-3-one;
(4aR,8aR)-6-[4-[[4-(trifluoromethyl)phenyl]methyl]piperidine-1-carbonyl]-4,4a,5,7,8,8a-hexahydropyrido[4,3-b][1,4]oxazin-3-one; and
rac-(4aS,8aS)-6-[4-(2-methylallyl)piperidine-1-carbonyl]-4,4a,5,7,8,8a-hexahydropyrido[4,3-b][1,4]oxazin-3-one (CAS 1941372-36-6).

3. The compound of formula (Ia) or (Ib) according to claim 1 or 2, or a pharmaceutically acceptable salt thereof, wherein A is azetidine or 7-azaspiro[3.5]nonan-7-yl and R^{A} is hydrogen.

4. The compound of formula (Ia) or (Ib) according to any one of claims 1 to 3, or a pharmaceutically acceptable salt thereof, wherein B is phenyl substituted with R¹, R² and R³.

5. The compound of formula (Ia) or (Ib) according to any one of claims 1 to 4, or a pharmaceutically acceptable salt thereof, wherein:
L is selected from a covalent bond, —CHR^{L}—, —CH₂CH₂—, —O—, —OCH₂—, and —CH₂O—; and
R^{L} is hydrogen or halo-C₆-C₁₄-aryl.

6. The compound of formula (Ia) or (Ib) according to any one of claims 1 to 4, or a pharmaceutically acceptable salt thereof, wherein L is selected from a covalent bond, —O—, —CH₂—, —CH₂CH₂—, and —CH₂O—.

7. The compound of formula (Ia) or (Ib) according to any one of claims 1 to 4, or a pharmaceutically acceptable salt thereof, wherein L is a covalent bond or —O—.

8. The compound of formula (Ia) or (Ib) according to any one of claims 1 to 7, or a pharmaceutically acceptable salt thereof, wherein R¹ is selected from halogen, C₁-C₆-alkyl, halo-C₁-C₆-alkyl, halo-C₁-C₆-alkoxy, hydroxy-C₁-C₆-alkyl, C₆-C₁₄-aryloxy, C₆-C₁₄-aryl, and C₃-C₁₀-cycloalkyl, wherein said C₃-C₁₀-cycloalkyl, C₆-C₁₄-aryloxy and C₆-C₁₄-aryl are substituted with 1-2 substituents that are independently selected from halogen and halo-C₁-C₆-alkyl.

9. The compound of formula (Ia) or (Ib) according to any one of claims 1 to 7, or a pharmaceutically acceptable salt thereof, wherein R¹ is selected from C₆-C₁₄-aryloxy, halo-C₁-C₆-alkyl and C₃-C₁₀-cycloalkyl substituted with halo-C₁-C₆-alkyl.

10. The compound of formula (Ia) or (Ib) according to any one of claims 1 to 7, or a pharmaceutically acceptable salt thereof, wherein R¹ is selected from phenoxy, CF₃ and (trifluoromethyl)cyclopropyl.

11. The compound of formula (Ia) or (Ib) according to any one of claims 1 to 10, or a pharmaceutically acceptable salt thereof, wherein R² is selected from hydrogen and halogen.

12. The compound of formula (Ia) or (Ib) according to any one of claims 1 to 10, or a pharmaceutically acceptable salt thereof, wherein R² is hydrogen or fluoro.

13. The compound of formula (Ia) or (Ib) according to any one of claims 1 to 12, or a pharmaceutically acceptable salt thereof, wherein R³ is hydrogen.

14. The compound of formula (Ia) or (Ib) according to claim 1 or 2, or a pharmaceutically acceptable salt thereof, wherein:
A is a 3-14 membered heterocycle;
B is C₆-C₁₄-aryl substituted with R¹ and R²;
L is selected from a covalent bond, —CH₂CH₂—, —CHR^{L}—, —O— and —CH₂O—;
R^{L} is hydrogen or halo-C₆-C₁₄-aryl;
R¹ is selected from halogen, C₁-C₆-alkyl, halo-C₁-C₆-alkyl, halo-C₁-C₆-alkoxy, hydroxy-C₁-C₆-alkyl, C₆-C₁₄-aryloxy, C₆-C₁₄-aryl, and C₃-C₁₀-cycloalkyl, wherein said C₃-C₁₀-cycloalkyl, C₆-C₁₄-aryloxy and C₆-C₁₄-aryl are substituted with 1-2 substituents that are independently selected from halogen and halo-C₁-C₆-alkyl; and
R² is selected from hydrogen and halogen.

15. The compound of formula (Ia) or (Ib) according to claim 1, or a pharmaceutically acceptable salt thereof, wherein:
A is a 3-14 membered heterocycle;
B is C₆-C₁₄-aryl substituted with R¹ and R²;
L is a covalent bond or —O—;
R¹ is selected from C₆-C₁₄-aryloxy, halo-C₁-C₆-alkyl and C₃-C₁₀-cycloalkyl substituted with halo-C₁-C₆-alkyl; and
R² is hydrogen or halogen.

16. The compound of formula (Ia) or (Ib) according to claim 1, or a pharmaceutically acceptable salt thereof, wherein:
A is azetidine or 7-azaspiro[3.5]nonan-7-yl;
B is phenyl substituted with R¹ and R²;
L is selected from a covalent bond, —CH₂— or —O—;
R¹ is selected from phenoxy, CF₃ and (trifluoromethyl)cyclopropyl; and
R² is hydrogen or fluoro.

17. The compound of formula (Ia) or (Ib) according to any one of claims 1 to 16, or a pharmaceutically acceptable salt thereof, selected from the compounds disclosed in Table 1.

18. The compound of formula (Ia) or (Ib) according to any one of claims 1 to 16, or a pharmaceutically acceptable salt thereof, selected from:
(4aR,8aR)-6-[3-[4-[1-(trifluoromethyl)cyclopropyl]phenyl]azetidine-1-carbonyl]-4,4a,5,7,8,8a-hexahydropyrido[4,3-b][1,4]oxazin-3-one 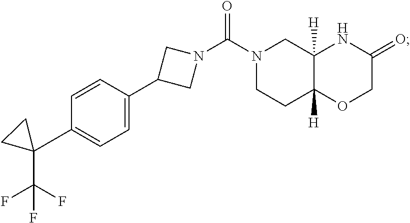 [SMILES: O=C1CO[C@@H]2CCN(C(=O)N3CC(c4ccc(C5(C(F)(F)F)CC5)cc4)C3)C[C@H]2N1]
(4aS,8aS)-6-(3-(4-phenoxyphenyl)azetidine-1-carbonyl)hexahydro-2H-pyrido[4,3-b][1,4]oxazin-3(4H)-one 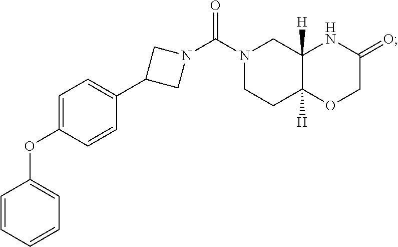 [SMILES: O=C1CO[C@H]2CCN(C(=O)N3CC(c4ccc(Oc5ccccc5)cc4)C3)C[C@@H]2N1]
and
(−)- or (+)-trans-6-[2-[2-Fluoro-4-(trifluoromethyl)phenoxy]-7-azaspiro[3.5]nonane-7-carbonyl]-4,4a,5,7,8,8a-hexahydropyrido[4,3-b][1,4]oxazin-3-one

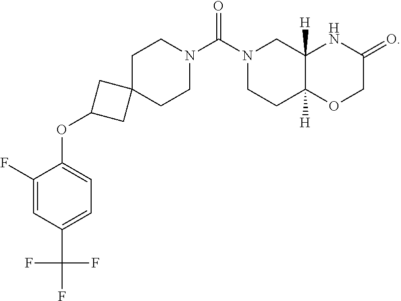 [SMILES: O=C1CO[C@H]2CCN(C(=O)N3CCC4(CC3)CC(Oc3ccc(C(F)(F)F)cc3F)C4)C[C@@H]2N1]

19. A process of manufacturing the compounds of formula (Ia) or (Ib) according to any one of claims 1 to 18, or pharmaceutically acceptable salts thereof, comprising:
reacting a first amine 4a,5,6,7,8,8a-hexahydro-4H-pyrido[4,3-b][1,4]oxazin-3-one (1) 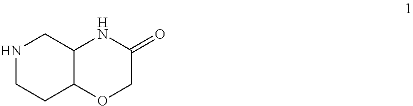 [SMILES: O=C1COC2CCNCC2N1]
with a second amine of formula 2, wherein A, L, and B are as defined in any one of claims 1 to 18 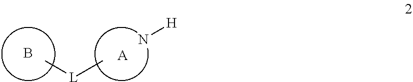
in the presence of a base and a urea forming reagent,
to form said compound of formula (Ia) or (Ib).

20. A compound of formula (Ia) or (Ib) according to any one of claims 1 to 18, or a pharmaceutically acceptable salt thereof, when manufactured according to the process of claim 19.

21. A compound of formula (Ia) or (Ib) according to any one of claims 1 to 18 and 20, or a pharmaceutically acceptable salt thereof, for use as therapeutically active substance.

22. A pharmaceutical composition comprising a compound of formula (Ia) or (Ib) according to any one of claims 1 to 18 and 20, or a pharmaceutically acceptable salt thereof, and a therapeutically inert carrier.

23. The use of a compound of formula (I) according to any one of claims 1 to 18 and 20, or a pharmaceutically acceptable salt thereof, or of a pharmaceutical composition according to claim 22 for the treatment or prophylaxis of neuroinflammation, neurodegenerative diseases, pain, cancer, mental disorders and/or inflammatory bowel disease in a mammal.

24. The use of a compound of formula (I) according to any one of claims 1 to 18 and 20, or a pharmaceutically acceptable salt thereof, or of a pharmaceutical composition according to claim 22 for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

25. A compound of formula (I) according to any one of claims 1 to 18 and 20, or a pharmaceutically acceptable salt thereof, or of a pharmaceutical composition according to claim 22 for use in the treatment or prophylaxis of neuroinflammation, neurodegenerative diseases, pain, cancer, mental disorders and/or inflammatory bowel disease in a mammal.

26. A compound of formula (I) according to any one of claims 1 to 18 and 20, or a pharmaceutically acceptable salt thereof, or of a pharmaceutical composition according to claim 22 for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

27. The use of a compound of formula (I) according to any one of claims 1 to 18 and 20, or a pharmaceutically acceptable salt thereof, for the preparation of a medicament for the treatment or prophylaxis of neuroinflammation, neurodegenerative diseases, pain, cancer, mental disorders and/or inflammatory bowel disease in a mammal.

28. The use of a compound of formula (I) according to any one of claims 1 to 18 and 20, or a pharmaceutically acceptable salt thereof, for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain, abdominal pain, abdominal pain associated with irritable bowel syndrome and/or visceral pain in a mammal.

29. A method for the treatment or prophylaxis of neuroinflammation, neurodegenerative diseases, pain, cancer, mental disorders, and/or inflammatory bowel disease in a mammal, which method comprises administering an effective amount of a compound of formula (I) according to any one of claims 1 to 18 and 20, or a pharmaceutically acceptable salt thereof, or of a pharmaceutical composition according to claim 22 to the mammal.

30. A method for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colon carcinogenesis, ovarian cancer, neuropathic pain, chemotherapy induced neuropathy, acute pain, chronic pain, spasticity associated with pain in a mammal, abdominal pain, abdominal pain associated with irritable bowel syndrome and/or visceral pain which method comprises administering an effective amount of a compound of formula (I) according to any one of claims 1 to 18 and 20, or a pharmaceutically acceptable salt thereof, or of a pharmaceutical composition according to claim 22 to the mammal.

31. The invention as described hereinbefore.
